(19) United States
(12) Patent Application Publication
(10) Pub. No.: US 2008/0075750 A1
(43) Pub. Date: Mar. 27, 2008

(54) METHODS FOR PRODUCING THREE-DIMENSIONAL TISSUE-ENGINEERED CARDIAC CONSTRUCTS AND USES REGARDING SAME

(75) Inventor: Robert E. AKINS JR., West Chester, PA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The Nemours Foundation, Jacksonville, FL (US)

(21) Appl. No.: 11/750,827

(22) Filed: May 18, 2007

Related U.S. Application Data
(63) Continuation-in-part of application No. 11/265,515, filed on Nov. 3, 2005, now abandoned.
Application No. 11/265,515 is a continuation of application No. 10/141,768, filed on May 10, 2002, now abandoned.
(60) Provisional application No. 60/290,026, filed on May 11, 2001.

(51) Int. Cl. A61F 2/00; A61K 35/00; A61K 38/00; A61P 9/00; C12P 1/00

(52) U.S. Cl. 424/423; 424/93.21; 424/93.7; 435/41; 514/12

(57) ABSTRACT

The invention relates generally to methods for producing a three-dimensional (3D) tissue-engineered cardiac construct and more specifically, a vascularized 3D tissue-engineered cardiac construct and uses regarding the same. The tissue-engineered cardiac constructs of the invention share the same physiological characteristics, such as contractile function, as in vivo intact cardiac tissue.

### CROSS-REFERENCE TO RELATED APPLICATIONS

This is a continuation-in-part application and claims priority to and benefit under 35 U.S.C. § 120 to U.S. application Ser. No. 11/265,515 filed, 3 Nov. 2005, which in turn is a continuation application and claims priority to and benefit under 35 U.S.C. § 120 to U.S. application 10/141,768, filed 10 May 2002, which claims priority to U.S. Provisional Application Ser. No. 60/290,026, filed May 11, 2001, the disclosures of which are herein expressly incorporated by reference in their entirety.

### STATEMENT REGARDING FEDERALLY-SPONSORED RESEARCH

This invention was made, at least in part, with U.S. government support under grant No. NAG9-1339, awarded by the National Aeronautics and Space Administration, Office of Biological and Physical Research. The U.S. government may have certain right in the invention.

### BACKGROUND

1. Field of the Invention

The invention relates generally to methods for producing a three-dimensional (3D) tissue-engineered cardiac construct and more specifically, a vascularized 3D tissue-engineered cardiac construct and uses regarding the same. The tissue-engineered cardiac constructs of the invention share the same physiological characteristics, such as contractile function, as in vivo intact cardiac tissue.

2. Related Art

The heart is a complex, muscular organ made up of multiple cell types organized in precise architectures. The correct organization and coordination of the cells in the heart are requisite for efficient contractile activity. The congenital absence of tissue-like structure or the disruption of appropriate tissue organization can be seriously debilitating. The clinical problem is substantial: significant cardiac malformations are found in nearly 14 of every 1,000 live births, and many people born with intact hearts subsequently incur damage as a result of disease, infection, or poor coronary circulation. Unfortunately, the heart's ability to regenerate is severely limited, and when circulatory function is diminished because of lost or absent cardiac structures, surgical intervention may be the only recourse to correct the deficiency. Moreover, there is a severe shortage of donor tissue available for cardiac reconstructions and transplantations. The application of tissue engineering methods wherein single donor organs could be used to produce multiple biosynthetic implants would greatly alleviate this shortage.

Biosynthetic constructs have been described that would replace or augment bone, cartilage, kidney, liver, neuronal tissue, and skin. Thus far, however, few advances in the engineering of cardiac tissue have been reported. The use of biosynthetic pulmonary valves in sheep has been previously demonstrated, and there have been several reports of cell transplantation studies in which a variety of cell types, including SV40-transformed cell lines, C2C12 myoblasts, isolated cardiomyocytes, skeletal myoblasts, and embryonic stem cells have been directly implanted into mammalian hearts. The results of these cell implantation studies clearly demonstrate the potential for implanted cells to incorporate into existing cardiac structures; however, the injection of cell suspensions may be of little benefit in cases where the local cardiac structure is missing or seriously damaged. The ability to establish the proper alignment and distribution of cardiac cells de novo, either within the context of damaged tissue or prior to implantation of biosynthetic constructs, would allow cell replacement therapies to be applied to the treatment of numerous cardiac defects. Unfortunately, the factors that control the establishment and maintenance of cardiac form are not clearly understood, and the ability to restore tissue level, cardiac organization ex vivo to disordered mixtures of cells has not been described.

### SUMMARY OF THE INVENTION

The invention satisfies the above needs by providing methodologies for producing a vascularized 3D cardiac construct that re-establish aspects of tissue morphology de novo. With the benefit of 3D, vascularized tissue-like organization, bioreactor-derived cultures may prove exceedingly useful in cell biological research and in tissue engineering.

In one aspect of the invention, a method for producing a three-dimensional vascularized cardiac construct is provided where cardiac cells are cultured in a bioreactor vessel containing a support under appropriate conditions to facilitate cell growth on the support, an effective amount of a first biologically active agent to the cardiac cell culture to facilitate cardiac cell migration is added to the bioreactor, the cells differentiate and organize into a three-dimensional construct, and an effective amount of a second biologically active agent is added to the three-dimensional cardiac cell construct to promote vascularization of the three-dimensional cardiac construct. In another aspect, the cardiac cells may be one or more mammalian cells such as cardiomyocytes, endocardial cells, cardiac adrenergic cells, cardiac fibroblasts, vascular endothelia cells, smooth muscle cells, cardiac progenitor cells, and stem cells.

In yet another aspect, the appropriate culturing conditions in said culturing step includes culturing the cardiac cells in the presence of a serum-free media. Further, the support may be a material such as sutures, meshes, foams, gels, ceramics, acellularized extra-cellular matrix material. Further, the suture may be fabricated from materials such as silk, polypropylene, polyamide, polyvinylidene, polyester, polyether, polydioxanone, nylon, linen, cotton, plain gut, chromic gut, poliglecaprone, polyglactin, polylactide, collagen, or naturally occurring protein, and any combination thereof. Additionally, the cardiac cells may coat the suture at a density of about 1×10⁶ cells/ml.

In one aspect of the invention, the first biological active agent may be at least one such as bone morphogenetic protein 2 (BMP 2) and ana modulator of notch signaling. Moreover, the second biological active agent may be at least one compound such as FGF, bFGF, acid FGF (aFGF), FGF-2, FGF-4, EGF, PDGF, TGF-betal, angiopoietin-1, angiopoietin-2, PlGF, VEGF, and any combination thereof.

In another aspect of the invention, a method for treating a subject afflicted with cardiac damage is provided where a three-dimensional, vascularized cardiac construct organized on a support is provided to the patient and the three-dimensional vascularized cardiac construct may be implanted in the subject. Furthermore, the three-dimensional cardiac construct may be treated with an effective amount of a composition comprising a biological active agent prior to implantation into the subject. The composition may be an immediate release composition capable of facilitating vascularization and integration of the three-dimensional cardiac construct into the in vivo cardiac tissue of the subject. Additionally, the composition may be a time release composition capable of facilitating vascularization and integration of the three-dimensional cardiac construct into the in vivo cardiac tissue of the subject.

Another aspect of the invention is related to a three-dimensional vascularized cardiac construct, which may include cardiac cells, and a support where the cardiac cells may be arranged on the support at a density of about 1×10⁶ per ml and may form a three-dimensional vascularized cardiac construct having physiological characteristics similar to intact it vivo cardiac tissue. Moreover, the cardiac construct may be genetically modified to produce one or more gene products having at least one ability such as enhancing the growth of seeded cells, enhancing migration, enhancing cardiac function, facilitating angiogenesis, and reducing the likelihood of thrombus formation.

In another aspect, the three-dimensional cardiac construct may be coated with an effective amount of a biological active agent. In particular, the biological agent may be capable of promoting angiogenesis. The biological agent may be at least one compound such as FGF, bFGF, acid FGF (aFGF), FGF-2, FGF-4, EGF, PDGF, TGF-betal, angiopoietin-1, angiopoietin-2, PlGF, VEGF, and any combination thereof. Also, the biological agent may be an antibiotic.

### BRIEF DESCRIPTION OF THE DRAWINGS

The accompanying drawings, which are included to provide, a further understanding of the invention, are incorporated in and constitute a part of this specification, illustrate embodiments of the invention and together with the detailed description serve to explain the principles of the invention. No attempt is made to show structural details of the invention in more detail than may be necessary for a fundamental understanding of the invention and various ways in which it may be practiced.

FIG. 1 is a schematic illustration of a cross-sectional representation of the tissue engineered cardiac constructs in accordance with embodiments of the invention showing critical features of the tissue-engineered cardiac constructs.

FIG. 2 illustrates the similarities between bioreactor cultured and plate cultured cells. Analysis of cells cultured in HARV bioreactors vs. cells cultured on control plates. Both systems show similar levels of cell attachment, glucose utilization, proportion of cardiac myocytes to other cell types as measured by myosin:DNA and f-Actin:DNA ratios, and intermediary metabolic enzyme activities.

FIG. 3 is a schematic depicting cells within a spinning bioreactor. Within the bioreactor, the cells will “fall” in a circular motion with a diameter dependent on the speed of bioreactor rotation. At the proper speed, this diameter is effectively zero, causing the cells to be suspended in a relatively motionless position within the growth medium.

FIGS. 4A-4B shows a primary cardiac cell culture in a bioreactor such as a HARV bioreactor at 6 days post inoculation. The cells attached to separate microcarriers interact together to form an aggregate structure surrounding the microcarriers. FIG. 4B shows an electron micrograph of microcarrier aggregates having a lower layer consisting of cardiomyocytes, a surrounding layer of extracellular matrix (ECM) and an external layer of cardiac endothelium (arrows indicate the surface of the aggregate). This tissue-like structure is similar to the cellular structure seen in cardiac tissue in vivo.

FIGS. 5A-5B shows a distribution of cells in monolayer and three dimensional (3D) culture at the light microscopic level. Parallel cultures of neonatal rat cardiac cells were prepared on fibronectin-coated, polystyrene surfaces and allowed to progress for 6 days prior to observation. (A) Distribution of cells cultured by traditional methods on a polystyrene surface. The larger arrow indicates elongate myocytes, and the smaller arrow indicates a region of nonmyocytes. The various cell types were randomly oriented and distributed in a patchwork pattern across the surface. (B) Typical culture of cells grown in three dimensions using low shear bioreactors and polystyrene beads. The larger arrow indicates a mass of cells located between two beads, and the smaller arrow indicates a thinner layer of cells along a bead surface. Although 3D masses of cells were clear, the relative distribution of myocytes and nonmyocytes was not easily visualized in 3D cultures at the light microscopic level. Bar=15 μM.

FIGS. 6A-6C depict scanning electron microscopic observations of cells cultured in three dimensions on fibronectin-coated polystyrene beads. (A) Typical cluster of beads and cells formed during 6 days of suspension culture. The entire surface of the cluster was encased in a layer of covering-cells exhibiting a “cobblestone-like” morphology consistent with their identification as endothelial cells. Bar=100 μm. (B) Similar cluster at higher magnification in which a split generated during postfixation processing in the endothelial-like cells revealed elongate, “myocyte-like” cells (arrow) lying along the bead surface. Bar=10 μm. At still higher magnification, C shows a view looking edge-long at a endothelial-like cell. Visible beneath this layer is a layer of matrix material (arrow) deposited as the cells modified the extracellular milieu of the aggregates. Bar=10 μm.

FIGS. 7A-7B shows scanning electron microscopic observation of cells cultured in three dimensions on fibronectin-coated collagen thread. Oriented collagen (type I, Organogenesis, Inc.) threads were used as an alternative to polystyrene as a support scaffold for cultures of neonatal rat cardiac cells grown in bioreactors for 6 days. Two different views of such cultures are shown. Bar=10 μm. (A) Region of a thread in which the layer of covering cells was mostly intact. The arrow indicates an endothelial-like cell. (B) Corresponding region in which the covering layer has been stripped away during processing. Elongate, myocyte-like cells were seen oriented along the long axis of the thread. The arrow indicates a region of cell junction that is resembling the intercalated discs seen between myocytes in vivo.

FIG. 8 shows a transmission electron micrograph of a cardiac cell culture grown on an oriented collagen thread. Thin sections of material from three dimensional cultures grown for 6 days on oriented collagen threads were stained with lead citrate and uranyl acetate to visualize ultrastructural organization. Multiple layers of cellular and extracellular material were clearly evident. An endothelial layer (EN) can be seen at the culture to medium interface (indicated by triangular arrowheads). Moving toward the collagen support scaffold, this vas followed by a layer of extracellular matrix (ECM) of variable thickness and a cardiomyocyte later (CM). The cells in the cardiomyocyte layer were oriented along the long axis of the collagen thread with clearly organized sacromeres (S) appearing above and below myonuclei (N). In addition, structures resembling adrenergic nerve termini were seen (AD) interacting with cardiomyocytes. These results confirmed the layered organization observed in the scanning electron microscope and demonstrate that cardiac cells can form 3D-tissue-like architectures in vitro. Bar=1 μm.

FIGS. 9A-9B show transmission electron micrographs of junctional complexes formed in three dimensional (3D) culture on oriented-collagen threads. As in FIG. 5, cells were grown on oriented collagen fibers for 6 days prior to collection. Apposed cells in the cardiomyocyte layer of 3D aggregates formed cellular junctions characteristic of cardiac cells in vivo. (A) Typical adherens junction (arrow) with corresponding z-line material in each cell (z). The periphery of the two cells is indicated by triangular arrowheads. Bar=1 μm (B) shows a typical cell periphery with gap junctions (arrows) clearly evident. Bar=200 nm.

FIG. 10 shows two 3D aggregates stained for MyHC (Panel I) and for vimentin and DNA (Panel II). Nuclear staining is clearly seen outside the MyHC-positive cells (arrows). These cells are vimentin positive, non-muscle cells.

Stained for vimentin (green using MAb V9) and DNA (blue using Hoechst 33258) showing surface layer of non muscle cells.

FIG. 11 shows Real time PCR data for BMP 2, Notch 1 and Notch 2 receptors, Jagged 1 and Notch ligand, and Notch downstream targets HERP2 and Hes 1.

FIG. 12 shows the Western blot data Jagged 1, Notch 1 cleaved ICD, and BMP 2 with respect to Troponin T loading controls. Relative protein levels as measured by band absorbance normalized to Troponin levels.

FIG. 13 shows a comparison of control and Noggin treated bioreactor cultures 6 days post inoculation. Control cultures show tissue-like aggregates of microcarrier supports with visible thick structure between them (indicated by arrows). Noggin treated culture only shows thin connections between about 2-3 microcarrier supports without thick structural buildup or no connection at all.

### DETAILED DESCRIPTION OF THE INVENTION

It is understood that the invention is not limited to the particular methodology, protocols, and reagents, etc., described herein, as these may vary as the skilled artisan will recognize. It is also to be understood that the terminology used herein is used for the purpose of describing particular embodiments only, and is not intended to limit the scope of the invention. It is also noted that as used herein and in the appended claims, the singular forms “a,” “an,” and “the” include the plural reference unless the context clearly dictates otherwise. This, for example, a reference to “a construct” is a reference to one or more constructs and equivalents thereof known to those skilled in the art.

Unless defined otherwise, all technical and scientific terms used herein have the same meaning as commonly understood by one of ordinary skill in the art to which the invention pertains. The embodiments of the invention and the various features and advantageous details thereof are explained more fully with reference to the non-limiting embodiments and/or illustrated in the accompanying drawings and detailed in the following description. It should be noted that the features illustrated in the drawings are not necessarily drawn to scale, and features of one embodiment may be employed with other embodiments as the skilled artisan would recognize, even if not explicitly stated herein.

Moreover, provided immediately below is a “Definition” section, where certain terms related to the invention are defined specifically. Particular methods, devices, and materials are described, although any methods and materials similar or equivalent to those described herein can be used in the practice or testing of the invention. All references referred to herein are incorporated by reference herein in their entirety.

### Definitions

“Allogenic,” as used herein, generally refers to an allogenic cell or tissue that originates from or is derived from a donor of the same species as the recipient.

“Angiogenesis” as used herein, generally refers to the growth of blood vessels in the two or three dimensional tissue-engineered constructs in response to stimuli, particularly in response to administration of an effective amount of an angiogenic factor.

“Angiogenic factor,” as described herein, may include natural and recombinant forms of a variety of peptides, e.g., growth factors and related molecules which are able to promote endothelial and smooth muscle proliferation leading to the formation of new blood vessels (angiogenesis) when administered to the bioreactor cell culture. Alternatively, the two or three dimensional tissue-engineered cardiac constructs may be coated with the desired angiogenic factor prior to implantation in a subject or the tissue and vessels surrounding the implanted cardiac construct in the subject may be treated with the desired angiogenic factor. Exemplary growth factors include fibroblast growth factor (FGF), basic FGF (bFGF), vascular endothelial growth factor (VEGF), epidermal growth factor (EGF) and platelet-derived growth factor (PDGF).

“Mammal,” as used herein, includes animals and humans. Thus, when referring to processes such as harvesting tissue from an animal, it is intended that the animal can be a human. Although at times reference may be made herein to “an animal or human,” this is not intended to imply that the term “animal” does not include a human.

“Autologous,” as used herein, generally refers to an autologous cell or tissue that originates or is derived from the recipient.

“Biologically Active Agent,” as used herein, generally refers to any naturally occurring or synthetic compound that is capable of inducing a change in the phenotype or genotype of a cell, tissue, organ, or organism when contacted with the cell, tissue, organ or organism. For example, the compound may enhance vascularization, cell survival, cell proliferation, cell differentiation, tissue formation; and compounds may inhibit fibrosis, inflammation, de-differentiation and tumorigenesis. The compounds may be secreted from the cells or exogenous compounds may he added to the cell or tissue culture media so as to supply the compound. The compound may include, for example, local chemical mediators, such as small molecule therapeutics (e.g., peptides), cell growth factors such as fibroblast growth factor (FGF), epithelial growth factor (EGF), vascular endothelial growth factor (VEGF) and hapatocyte growth factor (HGF); proteins such as insulin, somatototropin, somatomedin, adrenocorticotropic hormone (ACTH), parathyroid hormone (PTH) and thryroid-stimulating hormone (TSH); glycoproteins, amino acid derivatives such as somatostatin, varopressin, TSH releasing factor, and steroid such as cortisol, estradiol and testosterone.

The phrases “conditions suitable for growth” or “appropriate cell culture conditions” for a suitable cell type, as used herein, generally refers to an environment with conditions of temperature, pressure, humidity, nutrient and waste exchange, and gas exchange that are permissive for the survival and reproduction of the cells. With respect to any particular type of cell, an environment suitable for growth may require the presence of particular nutrients or growth factors needed or conducive to the survival and/or reproduction of the cells.

“Microcarrier Support,” as used herein, generally refers to any materials that are suitable for supporting cell growth, migration and differentiation and may include sutures, meshes, foams, gels, ceramics, acellularized extra-cellular matrix material, and the like. If a suture material is used for the support, the suture material is preferably made of silk, polypropylene, polyamide, polyvinylidene, polyester, polyether, polydioxanone, nylon, linen, cotton, plain gut, chromic gut, poliglecaprone, polyglactin, polylactide, collagen, or naturally occurring protein, and the like, and may include a combination of these materials. Furthermore, the microcarrier support may also include materials such as degradable or non-degradable polymers.

“Microcarrier Support Coating,” as used herein, generally refers to any coating that will facilitate cell attachment. The coating may be applied to at least one surface of the support. The coating may be, for example, cells, proteins, protein fragments, peptides, small molecules, lipid bilayers, metals and self-assembled monolayers.

“Polypeptide,” as used herein, refers to a linear series of amino acid residues connected to one another by peptide bonds between the alpha-amino group and carboxy groups of adjacent amino acid residues. Additionally covalent bonds between portions of the peptide are also present to restrain the conformation of the molecule, such as amide and disulfide bonds. When used herein, “protein” also refers to a linear series of amino acid residues connected one to the other as in a peptide. The term “synthetic peptide” means a chemically derived chain of amino acid residues linked together by peptide bonds that is free of naturally occurring proteins and fragments thereof. The polypeptides of the invention may be a naturally purified product, or a product of a chemical synthetic-procedure, or produced by recombinant techniques from a prokaryotic or eukaryotic host.

“Primary Culture Cells,” as used herein, generally refer to cells that are not fully differentiated but may have the capacity to either become more fully differentiated or to give rise to a cell (or cells) that is able to further differentiate. The primary culture cell may be capable of giving rise to one or more different cell types. More particularly, primary culture cells may be cells that either have a native capacity for differentiation into 3D cardiac constructs or that the cells may be manipulated into forming 3D cardiac constructs. Accordingly, small samples of autologous, allogenic or xenogeneic donor cells may be used far constructs.

The cell types that may be used to generate that 3D cardiac construct of the invention may include, but are not limited to, cardiomyocytes, endocardial cells, cardiac adrenergic cells, cardiac fibroblasts, vascular endothelial cells, smooth muscle cells, stem cells, cardiac progenitor cells, and myocardial precursor cells. Depending on the application of the two or three dimensional cardiac construct and the type of cardiac tissue material that is desired, the above types of cells may be used independently or combined with one another. In one embodiment, the two or three dimensional cardiac constructs may be composed of primary tissue isolates from the heart. Alternatively, cells such as non-immunogenic universal donor cell lines or stem cells may be used so long as they can be manipulated to farm 3D cardiac constructs.

“Subject,” as used herein, includes individuals who require intervention or manipulation due to a disease state, treatment regimen or experimental design. Furthermore, the term “subject” includes animals and humans.

“Tissue-engineered construct,” as used herein, generally refers to a two or three dimensional mass of living mammalian tissue produced primarily by growth in vitro. The construct may include one or more types of cells or tissues. For example, the tissue-engineered construct may be made up of myocytes cultured in conjunction with other cell types, such as endocardial cells, vascular smooth muscle cells, vascular endothelium, fibroblast, and adrenergic cells, or various subsets of those cell typos. The term also encompasses a two or three dimensional mass of living mammalian tissue produced at least in part by growth in vivo on a substrate. More particularly, tissue-engineered constructs may include two or three dimensional tissue which share critical structural and functional characteristics with intact cardiac tissue, such as distinctive multicellular organization and oriented contractile function.

“Xenogeneic,” as used herein generally refers to a cell or tissue that originates from or is derived from a donor of a different species than the recipient.

The invention relates generally to methods and products of use in the field of tissue engineering and replacement tissues and organs. In particular, in one embodiment the invention provides methods for producing 3-dimensional (3D) tissue-engineered cardiac constructs by using a bioreactor system and more particularly, to produce a vascularized 3D tissue-engineered cardiac construct. In a specific aspect, the cardiac constructs share critical structural and functional characteristics with intact cardiac tissue, such as distinctive multicellular organization and oriented contractile function.

According to an embodiment of the invention, the tissue engineered cardiac constructs may be implanted into a subject for the treatment of conditions involving tissue damage or dysfunction in accordance with standard surgical procedures. In particular, the tissue-engineered cardiac construct may be implanted directly into, for example, damaged (or other) areas of a heart during heart surgery in order to augment contractile function. In one embodiment, heart disease would be treated by surgically placing one or more tissue engineered cardiac constructs across a non-functional region of the heart bridging two healthy regions. The suture would be placed along the axis of contraction for that region of the heart. After implantation, cells from the subject may migrate into the tissue in vivo, thereby complementing the seeded cell population. Such a placement would help to re-establish the correct propagation of the contractile signal and would provide contractile elements. The cells of the implanted tissue engineered cardiac construct may be derived from allogenic, autologous or xenogeneic donor cells and/or tissues.

In additional embodiment, the tissue-engineered cardiac constructs may be used in basic research for testing mid characterizing pharmacologically active compounds. The use of human tissue-equivalents in vitro to test the safety, efficacy, and mechanism of action of potential therapeutic agents is clearly desirable, especially since current methods of analysis fall short. In addition, studying human cardiac-tissue equivalents could dramatically expand the current understanding of cardiac cell biology, cardiac physiology, and the relationships between cardiac function and structure during embryogenesis and cardiac remodeling.

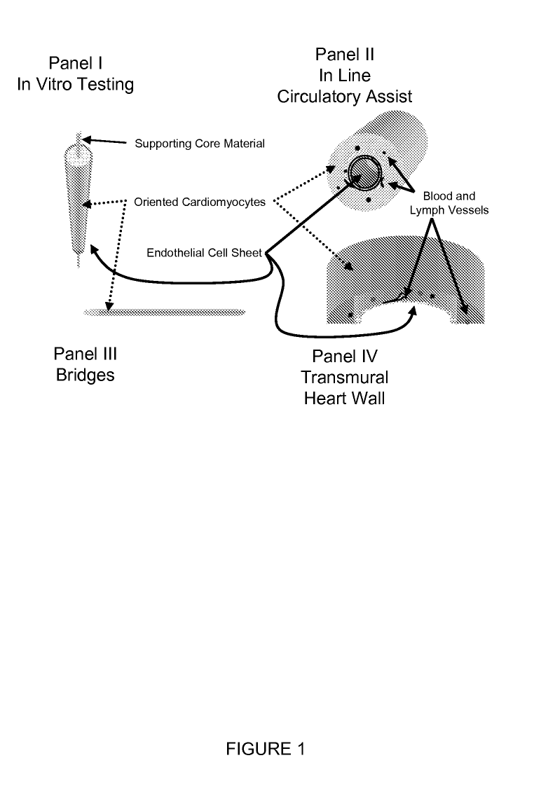

In general, the cardiac constructs of the present invention may be produced according to the methods described below. Typically, these methods involve seeding a bioreactor vessel with primary cardiac cells and culturing the bioreactor vessel under conditions suitable for growth of the cells and for a period of time sufficient to produce a construct of desired thickness and/or properties. FIG. 1 depicts a schematic diagram of a tissue-engineered cardiac construct produced by the methods of the invention. Panel I of FIG. 1, shows myocytes arranged parallel to a core material and lined with an EC sheet mimicking the blood interface. Panel III of FIG. 1, shows a a cardiomyocyte tube with an EC sheet lining, blood vessels and lymph vessels. Panel II of FIG. 1, shows strips of specialized myocytes to bridge conduction tissue, atrioventricular nodes, or sino-atrial nodes. Panel IV of FIG. 1, shows a 3D transmural patch with myocytes oriented in sheets like native myocardium.

In a further embodiment, various growth conditions or biologically active compounds may be selected to enhance growth of the tissue-engineered cardiac construct and/or to stimulate development of desirable mechanical, physical, or biochemical properties. For example, the two or three dimension tissue-engineered constructs may be subjected to electrical stimulation under the appropriate conditions to stimulate angiogenesis. Further, the growth conditions may include the use of particular growth media, the application of mechanical, electrical and/or chemical stimuli. Additionally, biologically active compounds may be added to the growth media to facilitate angiogenesis, migration, organization, function or integration of the tissue-engineered cardiac construct. For example, the biologically active compounds may include antioxidants or cell-cycle inhibitors such as sirolimus.

In a particular embodiment, a method for promoting angiogenesis in the tissue engineered cardiac construct is provided. An amount of angiogenic factor sufficient to promote angiogenesis in the three dimensional tissue-engineered construct may be added to the bioreactor culture media, infra. Specifically, an angiogenic factor, such as VEGF, may be added to the bioreactor culture media at an amount in the range of 1 ng/ml to about 500 ng/ml, and more particularly, in an amount of 50 ng/ml. Moreover, the angiogenic factor may be added at all amount in the range of about 10 ng/ml to about 300 ng/ml, 20 ng/ml to about 200 ng/ml, about 30 ng/ml to about 100 ng/ml, and about 40 ng/ml to about 75 ng/ml.

Alternatively, the cell used to produce the tissue-engineered construct, such as the primary culture cells, may be genetically modified to contain a gene of interest as described below, and specifically a gene encoding an angiogenic factor.

Angiogenic factors suitable for use in the invention include a variety of known growth factors, such as FGF, bFGF, acid FGF (aFGF), FGF-2, FGF-4, EGF, PDGF, TGF-betal, angiopoietin-1, angiopoietin-2, placental growth factor (PlGF), VEGF and the like. The phrases “FGF polypepetide,” “VEGF polypeptide,” “EGF polypepetide,” and “PDGF polypeptide” may be defined to include all natural and recombinant forms of the full length proteins as well as fragments, analogs, mimetics, and other related molecules have similar or identical angiogenic activity. Generally, an effective amount of the angiogenic factor may be based upon the subject's body weight. In particular, the effective amount may be in the range of about 0.1 μg/kg body weight to about 100 mg/kg body weight, and more specifically, in the range of about 1 μg/kg of body weight to about 10 mg/kg of body weight.

In a further embodiment, the tissue-engineered constructs of the invention may be treated with any of a variety of biologically active agents prior to implantation into a subject. The agents may be saturated into the cardiac construct or bay be a time release formulation. In certain embodiments, these agents may be selected to enhance the properties of the construct following implantation, such as the ability of andogenous cells to populate the construct, to enhance the growth of seeded cells, to facilitate angiogenesis, to reduce the likelihood of thrombus formation and so on. Alternatively, the cell used to produce the tissue-engineered construct, such as the primary culture cells, may be genetically modified as described below to contain a gene of interest, and specifically a gene encoding a biologically active agent. The biologically active agents may include, but are not limited to thrombomodulators, such as heparin and low molecular weight heparin, agents that increase hemocompatability, growth factors, angiogenic factors, anti-coagulants and antibiotics. In particular embodiments, a pharmaceutical composition may be utilized which comprises at least one biologically active agent, such as an antibiotic. The pharmaceutical composition may be intended for treatment of the same condition as that being treated by implanting the tissue-engineered construct or for treatment for a different condition.

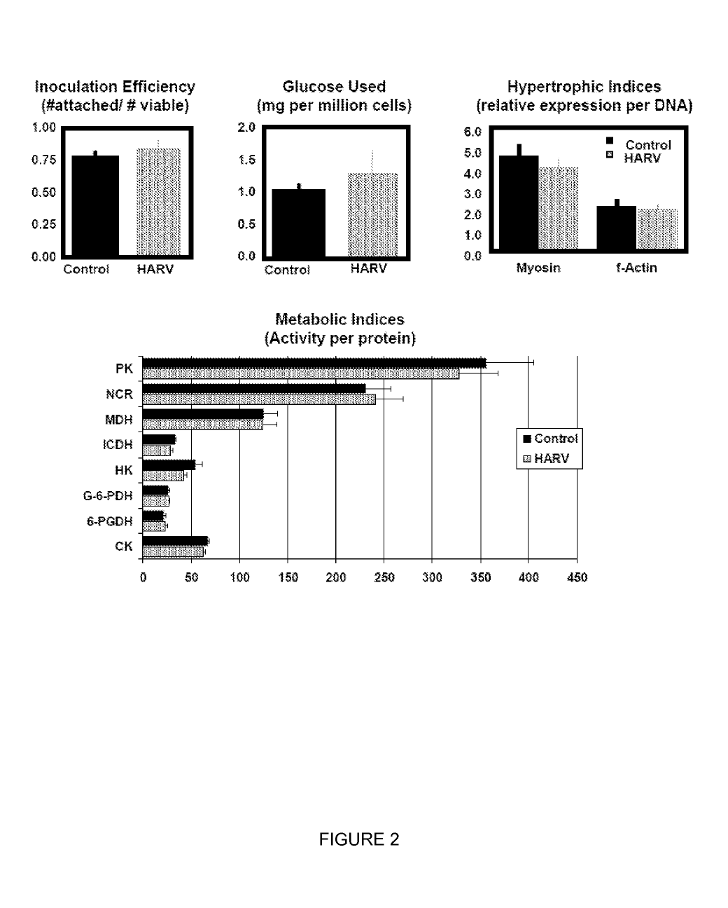

In general, the assembly of the cardiac constructs in the bioreactor system occurs in three steps: 1) adhesion 2) re-arrangement, and 3) remodeling. Of these, step 1 appears to be similar in bioreactors and 2D culture plates, whereas, steps 2 and 3 in the bioreactor differ from 2D culture plates. FIG. 2 illustrates the similarities between bioreactor cultures and plate cultures. Both systems show similar levels of cell attachment, glucose utilization, proportion of cardiac myocytes to other cell types as measured by myosin: DNA and f-Actin:DNA ratios, and intermediary metabolic enzyme activities.

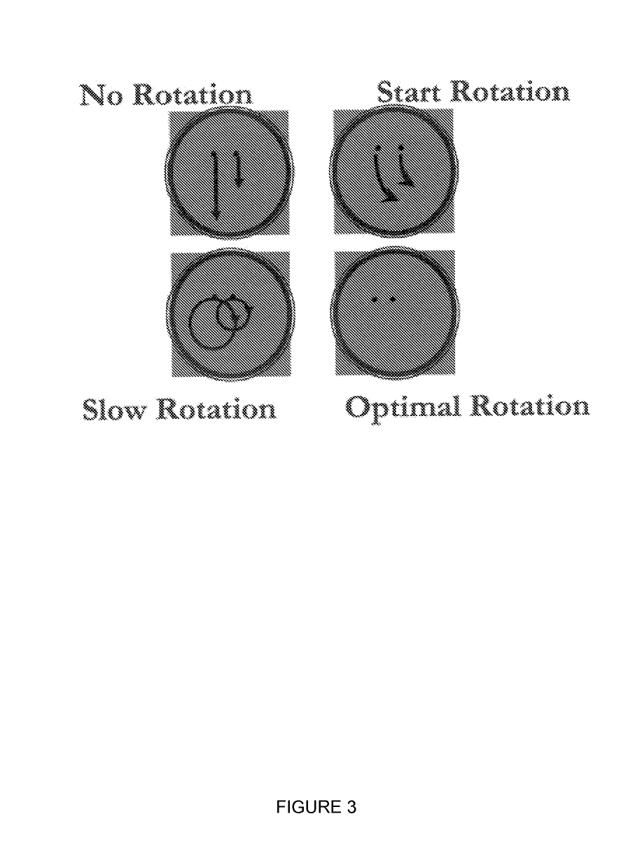

In one embodiment of the invention, isolated primary cardiac cells may be placed within a reactor vessel along with microcarrier supports to function as an attachment surface for the cardiac cells and rotated perpendicular to the gravitational plane. At the proper rotational speed, the gravitational acceleration is normalized with respect to the cells, which causes the cells to hang suspended within the culture media co-localized to each other in a state of simulated microgravity as shown in FIG. 3. Under these culture conditions in the bioreactor, the cardiac cell types maintain similar population levels and metabolic activity in direct comparison to cells grown in traditional 2D plate culture.

According to an embodiment of the invention, the primary culture cells may be derived from an animal or cell line of the same species as the intended recipient so that the resulting construct contains proteins that will be minimally antigenic and maximally compatible in the body. For example, if the construct is to be implanted into a human, the cells may be human cells. Alternatively, the cells of the construct may be derived from a xenogeneic donor. Although the general production of the tissue engineered cardiac construct involves culturing the developing tissue primarily in vitro, tissue engineered cardiac constricts produced at least in part by culturing the tissue in vivo are also within the scope of the invention.

Briefly, in traditional, 2D-culture methods the cells are confined to laterally associate on a flat surface beneath a layer of medium. As a result of the attachment dependence, contact inhibition, and low motility of cardiac cells, 2D culture methods result in a near monolayer of cells in a mosaic pattern. The distribution does not resemble the original tissue organization, and, although the population of cells in 2D cultures is representative of the population of cells in the tissue, 2D cultures do not accurately represent the tissue. Furthermore, it is known that the specific interactions of the multiple cell types present in cardiac cultures and the tertiary organization of the extracellular matrix dramatically affect myocardial cell phenotype. Some of the characteristics of cardiac cells cultured in non-tissue arrangements may not accurately reflect the characteristics of the same cells within the tissue's organization.

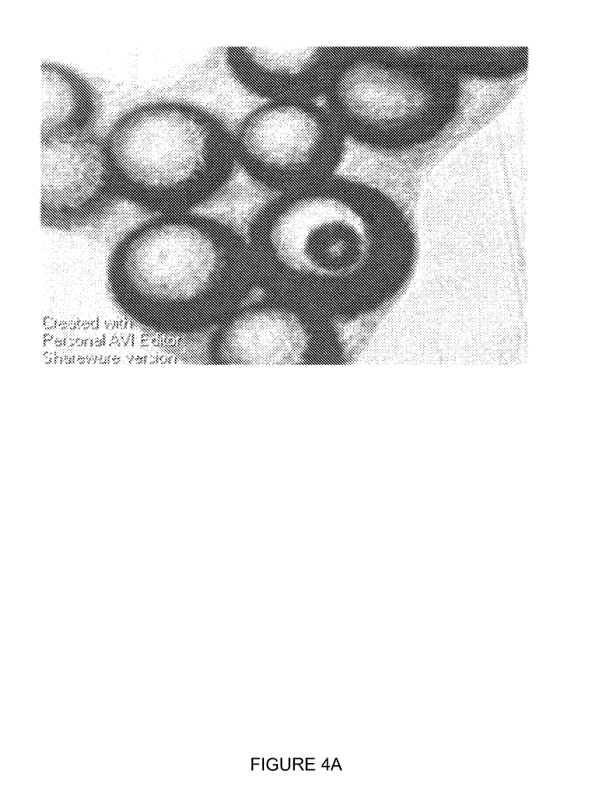

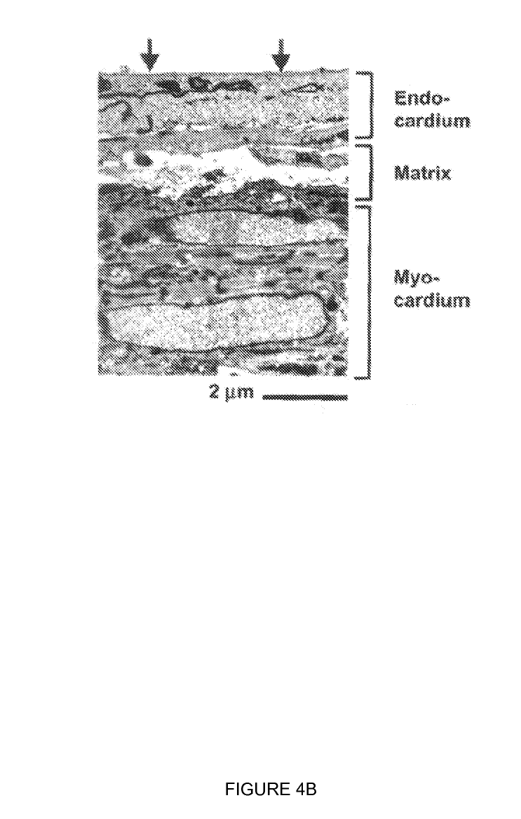

In direct contrasts, cardiac cells cultured in a bioreactor system show an organized aggregate structure. Electron microscopy of cardiac aggregates shows the formation of a layered structure. Specifically, the layered structure includes cardiomyocytes covered by a layer of extracellular matrix (ECM) which is surrounded by a layer of endothelium, a structure similar to that seen in normal intact, in vivo cardiac tissue as shown in FIGS. 4A-4B. Surprisingly, this tissue-like structure forms in the absence of any external cues from a scaffold or similar supplied source. Thus, formation of this tissue-like structure may depend upon the innate activities of the cardiac cells in the bioreactor culture environment.

In a particular embodiment, the ability of cells to distribute into tissue-like layers and the ability of cultures to express the cardiac extracellular matrix (ECM) may be a direct result of the specific cell interactions and migrations that are permitted because of the reduced shear levels and minimized gravitational effects in rotating bioreactors. The sheer stress may be a relatively low level, in a particular, about 0.49 dyne per cm². Additionally, the net-zero gravity vector of a rotated bioreactor may result in the more tissue-like placement of nuclei observed within myocytes. Organelles and cytoskeletal components of cardiac cells red redistribute during the early phases of cell culture, and gravity may influence nuclei to a basal location in cultures that are not clinostatically rotated. Alternatively, the differences in organelle distribution between bioreactor and traditional cultures may be related to the relative positions of cells and cell interactions in 3D, tissue-like architectures as opposed to 2D mosaic-like patterns.

In yet another embodiment, cell signaling pathways may govern the control of endothelial cell migration and as a direct result may play a part in the formation of the tissue-like aggregates within the bioreactor system of the invention. In particular bone morphogenic protein-2 (BMP 2), a secreted protein factor similar to TGF-β originally known for its functions in bone development, may be an important factor in cardiac development due to its role in cardiac cushion and valve formation. In order to form the thickened structures needed for these areas of the heart, BMP2 is important in the process of endothelial-mesenchymal transition (EMT), an endothelial cell migratory process in which endothelial cells move into the extracellular matrix layer beneath them and undergo a transition to a mesenchymal cell type for expansion and thickening of the extracellular matrix. Studies have shown that BMP2 or the presence of cardiomyocytes may be sufficient to induce endothelial cell migration and transition, and in the absence of either factor the cells did not migrate but rather remained in place. In addition, BMP 2 activity may be linked to regenerative activity in vivo, a process which inevitably requires endothelial/epithelial cell migration and may, thus demonstrate a continued function for BMP 2 after the completion of development.

In another embodiment, a second cell signaling pathways may play a part in the formation of the tissue-like aggregates, such as the Notch signaling pathway. Notch is a family of cell surface receptors which bind to certain membrane-bound ligands on neighboring calls; upon binding of these lights. Notch is then cleaved by the membrane protease γ-secretase, releasing an intracellular domain (ICD) which then travels to the nucleus, binding to Suppressor of Hairless (SuH)/CBF protein to induce production of proteins such as Hes and the Hes-related (HERP) families. Notch has many functions in development via its mediation of cell-cell interactions; in cardiac tissue it, like BMP 2, is associated with the EMT process and endothelia cell migration, as well as regenerative processes in various tissues. In addition to their own individual functions, the BMP 2 and Notch pathways are known to communicate with each other via crosstalk of their downstream mediators and targets, allowing each pathway to regulate and/or modify the signaling activity of each other. Members of both pathways show substantial increase in both mRNA and protein levels occurring within about a 24 hour period (see specific example 4, below). Disruption of BMP 2 activity by addition of the BMP antagonist, Noggin, may be sufficient to cause disruption in the formation of tissue-like aggregates in bioreactor cultured cells. This may suggest a role for BMP 2 in the formation of such aggregates under bioreactor conditions.

In another embodiment, endothelial cells (EC) are organized within 3D cardiac muscle constructs. Cardiac muscle tissues are complex assemblages comprising multiple cell types organized in precise structures. These structures determine the complex electrical and mechanical properties of the organ, provide interfaces for interactions between the hear and other tissues, and allow the requisite exchange of nutrients and wastes for proper tissue function. A critical feature of cardiac tissues is the distribution of ECs, which distribute in response to bioactive compounds and mechanical cues.

ECs occur in three general distributions within the heart: 1) as a sheet lining the endocardial surface and separated from the muscle proper by extracellular matrix, 2) as tubes lining coronary blood vessels and either closely associated with the muscle of the encompassing tissue or separated by intimal, medial, and advential tunics of varying thickness and cell/matrix composition, or 3) within lymphatic plexuses and vessels, which may also be either intimately associated with the surrounding tissue or separated. The location and density of blood and lymph vessels are critical for proper nutrition, perfusion, and fluid movement within the heart. Blood vessels also provide the interface for humoral factors coming into or going out of the heart, and, along with the endocardial surface, they protect the tissue from potentially damaging fluid shear and edema. Importantly, EC structures provide critical support to the heart without disrupting its electro-mechanical properties, which ultimately determine contractile function. Thus, controlling EC structure formation is critical to tissue engineering and regenerative therapies for the heart.

Bioreactor Systems

It is important that the bioreactor system used to produce the tissue-engineered cardiac constructs of the invention provide a low-shear environment. For example, suitable bioreactors for 3D culture include high-shear spinner-flasks or the use of gel matrices and low-shear clinostats like the NASA-designed vessels (e.g., High Aspect Ratio Vessel, HARV; Slow Turning Lateral Vessel, STLV, Hydrodynamic-Focusing Bioreactor, HFB) or clinostatically rotated FEP-bags. The low-shear environment found in NASA bioreactors (<0.52 dyne/cm² for the HARV) and in rotated Teflon®/fluorethylene polymer (FEP) bags are especially suitable for the culture of the tissue-engineered cardiac constructs of the invention.

In a specific embodiment, a High Aspect Ratio Vessel (HARV) may be used for growing cardiac tissue constructs. HARV bioreactors are rotating culture vessels that allow cells to be perpetually suspended in a low shear environment. The bioreactors may be operated in a humidified tissue culture incubator and have two machined Lexan (polycarbonate) discs. The discs are fitted together with an “O-ring” to form a cavity between them. One interior side of the HARV bioreactor module may be covered by a silicone membrane to allow gas exchange between the incubator environment and the bioreactor cavity. Cells and support surfaces may be inoculated into bioreceptor modules through the luerlock ports and the modules may be rotated to normalize the sedimentation effects of gravity until there is no visible movement of the dispersed contents relative to the bioreactor walls. This results in a quiescent fluid environment in which materials may be maintained in relative positions for extended period of time. To allow direct microscopic visualization of 3D construct without sampling, HARV bioreactors may replaced with FEP bags in some experiments.

In another embodiment, cell-supports may include polystyrene tissue culture plates and polystyrene microcarrier beads (Nunc.InterMed, Roskilde, Denmark) as well as oriented collagen fibers (Organogenesis, Canton Mass.). The culture plates and microcarrier beards may be shipped sterile from the manufacturer. The surface chemistries of the polystyrene plates and beads may be certified by the manufacturer to be the identical tissue culture grade.

Cell types that have been successful cultured using a HARV bioreactor include satellite cells, cells for polymer cartilage implants, small intestinal cells, colon carcinoma cells, ovarian tumor cells, rat heart cells, and rat ventricular cells. The types of cells that may be used in forming the human bio-active cardiac construct may include cardiomyocytes, endocardial cells, cardiac adrenergic cells, cardiac fibroblasts, vascular endothelial cells, smooth muscle cells, cardiac progenitor cells, and stem cells. Depending on the application of the tissue-engineered cardiac construct arid the type of cardiac tissue material that is desired, the above types of cells may be used independently or combined with one another.

The inoculation density depends upon the desired result. In a particular embodiment, the cell density is about 1×10⁶ cells/ml on 5 cm² support surface. Changes to this ratio alter the overall size and cellular thickness of the constructs prepared. The density of the cells will vary depending on certain variables such as, the exact type of cardiac tissue desired and the size and shape of the support. Generally, for most applications such as that for cardiac tissue on a suture support, a cell density ranging from about 0.5×10⁶ cells per cm² of support material surface area in 1 cc of medium to about 2.5×10⁶ cells per cm² of support material surface area in 1 cc of nutritive medium will be appropriate. The cells are introduced into the bioreactor as a suspension. Any suspension medium known to those skilled in the art may be used.

Materials that are suitable for microcarrier supports include sutures, meshes, foams, gels, ceramics, acellularized extra-cellular matrix material, and the like. If a suture material is used for the support, the suture material is preferably made of silk, polypropylene, polyamide, polyvinylidene, polyester, polyether, polydioxanone, nylon, linen, cotton, plain gut, chromic gut, poliglecaprone, polyglactin, polylactide, collagen, or naturally occurring protein, and the like, and may include a combination of these materials.

The rate of rotation of the bioreactor is a factor to consider. If the rate of rotation is too high, an undesirable centrifugal effect may be observed. The sedimentation of cells (or support material, etc.) in the bioreactor combined with the rotational movement of the medium results in the cell taking an elliptical path relative to the observer. By adjusting the rotation rate of the bioreactor, the dimensions of this elliptical path can be reduced so that the vessel, medium, and contents appear to maintain their relative relationships in a low-shear environment.

The bioreactor may be placed into an incubator with a controlled environment. The composition of the gas environment in the incubator is predicated by the selection of medium used. Specifically, the level of CO₂ depends upon the medium chosen, particularly the amount of sodium bicarbonate level in the medium. For example, AI-1medium containing about 2.45 mg/ml sodium bicarbonate may be used in about a 5% CO₂ environment. In a specific embodiment, a mixture of air with about 5% CO₂ may be used in conjunction with bicarbonate-buffered medium. Alternatively, air alone has been used in conjunction with a HEPES-buffered medium. The temperature of the incubator environment may be held at about 37° C., and the relative humidity of the incubator environment may be held at about 90%.

Gene Therapy

As described above, another embodiment of the present invention also involves the use of gene therapy applications, which may enhance the properties of the tissue-engineered construct during in vitro culturing and following implantation, such as the ability of endogenous cells to populate the construct, to enhance the growth of seeded cells, to enhance migration, to enhance function, to facilitate angiogenesis, to reduce infection, to reduce the likelihood of thrombus formation and so on. The tissue-engineered cardiac construct may include cells that are genetically engineered to produce one or more of the above listed biologically active agents or angiogenic factors. Gene therapy has been broadly defined as “the correction of a disease phenotype through the introduction of new genetic information into the affected organism” (Roemer et al., 208 Eur. J. Biochem. 211-25 (1992)). Two basic approaches to gene therapy have evolved: (1) ex vivo gene therapy and (2) in vivo gene therapy. In ex vivo gene therapy, cells are removed from a subject and cultured in vitro. A functional replacement gene is introduced into the cells (transfection) in vitro, the modified cells are expanded in culture, and then re-implanted in the subject. These genetically modified, re-implanted cells are reported to secrete detectable levels of the transfected gene product in situ (Miller, 76 Blood 271-8 (1990)) and Selden et al., 317 New Eng. J. Med. 1067-76 (1987)). The development of improved retroviral gene transfer methods (transduction) facilitates the transfer into and subsequent expression of genetic material by somatic cells (Cepko et al., 37 Cell 1053-62 (1984)). Accordingly, retrovirus-mediated gene transfer has been used in clinical trials to mark autologous cells and as a way of treating genetic disease (Rosenberg et al., 323 New Eng. J. Med 570-8 (1990); Anderson, 2 Human Gene Ther. 99-100 (1991)). Several ex vivo gene therapy studies in humans are reported (reviewed in Anderson, 256 Science 808-13 (1992) and Miller, 357 Nature 455-60 (1992)).

In in vivo gene therapy, target cells are not removed from the subject. Rather, the transferred gene is introduced into cells of the recipient organism in situ, that is, within the recipient. In vivo gene therapy has been examined in several animal models (reviewed in Felgner et al., 349 Nature 351-2 (1991)). Publications have reported the feasibility of direct gene transfer in situ into organs and tissues such as muscle (Ferry et al., 88 Proc. Natl. Acad. Sci. 8377-781 (1991); Quantin et al., 84 Proc. Natl. Acad. Sci. USA 2581-4 (1992)), hematopoietic stem cells (Clapp et al., 78 Blood 1132-9 (1991)), the arterial wall (Nabel et al., 244 Science 1342-4 (1989)), the nervous system (Price et al., 84 Proc. Natl. Acad. Sci. USA 156-60 (1987).) and lung (Rosenfeld et al., 252 Science 431-4 (1991)). Direct injection of DNA into skeletal muscle (Wolff et al., 247 Science 1465-8 (1990)), heart muscle (Kitsis et al., 88 Proc. Natl. Acad. Sci. USA 4138-42 (1991)) and injection of DNA-lipid complexes into the vasculature (Lim et al., 83 Circulation 2007-11 (1991); Ledere et al., 90 J. Clin. Invest. 936-44 (1992); Chapman et al., 71 Circ. Res. 27-33 (1992)) also have been reported to yield a detectable expression level of the inserted gene product(s) in vivo.

The delivery of an effective dose of the biologically active agent or angiogenic factor in situ depends on the efficiency of transfection (or transduction) as well as the number of target cells. Cardiac cell-based gene therapy, in particular, involves a relatively small area available in situ for receiving genetically modified cardiac cells. The delivery of an effective dose of biologically active agent or angiogenic factor in situ thus depends upon the total number of implanted cardiac cells.

In one embodiment of the invention, exogenous genetic material (e.g., a cDNA encoding a biologically active agent polypeptide or angiogenic factor polypeptide) is introduced into a syngeneic host cell ex vivo or in vivo by genetic transfer methods, such as transfection or transduction, to provide a genetically modified host cell. Various expression vectors (i.e., vehicles for facilitating delivery of exogenous genetic material into a target cell) are known to one skilled in the art.

Transfection refers to the insertion of nucleic acid into a mammalian host cell using physical or chemical methods. Several transfection techniques are known to those of ordinary skill in the art including; calcium phosphate DNA co-precipitation (Gene Transfer and Expression Protocols in Methods In Molecular Biology, Vol. 7 (E. J. Murray, ed., Humana Press) (1991)); DEAE-dextran; electroporation; cationic liposome-mediated transfection; and tungsten particle-facilitated microparticle bombardment (Johnston, 346 Nature 776-7 (1990)). Strontium phosphate DNA co-precipitation (Brash et al., 7 Mol. Cell. Biol. 2031-4 (1987)) may be a preferred transfection method.

In contrast, transduction refers to the process of transferring nucleic acid into a cell using a DNA or RNA virus. An RNA virus (i.e., a retrovirus) for transferring a nucleic acid into a cell is referred to herein as a transducing chimeric retrovirus. Exogenous genetic material contained within the retrovirus is incorporated into the genome of the transduced host cell. A host cell that has been transduced with a chimeric DNA virus (e.g., an adenovirus carrying a cDNA encoding a biologically active agent polypeptide or angiogenic factor polypeptide) will not have the exogenous genetic material incorporated into its genome, but will be capable of expressing the exogenous genetic material that is retained extrachromosomally within the cell.

Typically, the exogenous genetic material includes the heterologous gene (usually in the form of a cDNA comprising the exons coding for the biologically active agent or angiogenic factor) together with a promoter to control transcription of the new gene. The promoter characteristically has a specific nucleotide sequence necessary to initiate transcription. Optionally, the exogenous genetic material further includes additional sequences (i.e., enhancers) required to obtain the desired gene transcription activity. As used herein an enhancer is simply any non-translated DNA sequence which works contiguous with the coding sequence to change the basal transcription level dictated by the promoter. Specifically, the exogenous genetic material is introduced into the host cell genome immediately downstream from the promoter so that the promoter and coding sequence are operative linked so as to permit transcription of the coding sequence. A retroviral expression vector includes an exogenous promoter element to control transcription of the inserted exogenous gene. Such exogenous promoters include both constitutive and inducible promoters.

Naturally-occurring constitutive promoters control the expression of essential cell functions. As a result, a gene under the control of a constitutive promoter is expressed under all conditions of cell growth. Exemplary constitutive promoters include the promoters for the following genes which encode certain constitutive or housekeeping function: hypoxanthine phosphoribosyl transferase (HPRT), dihydrofolate reductase (DHFR) (Scharfmann et al., 88 Proc. Natl. Acad. Sci. USA 4626-30 (1991)), adenosine deaminase, phosphoglycerol kinase (PGK), pyruvate kinase, phosphoglycerol mutase, the beta-actin promoter (Lai et al., 86 Proc. Natl. Acad. Sci. USA 10006-10 (1989)), and other constitutive promoters known to those of skill in the art. In addition, many viral promoters function constitutively in eukaryotic cells. These include: the early and late promoters of SV40; the long terminal repeats (LTPs) of Moloney Leukemia Virus and other retroviruses; and the thymidine kinase promoter of Herpes Simplex Virus, among many others. Accordingly, any such constitutive promoters can be used to control transcription of a heterologous gene insert.

Genes that are under the control of inducible promoters are expressed only or to a greater degree, in the presence of an inducing agent, (e.g., transcription under control of the metallothionein promoter is greatly increased in presence of certain metal ions). Inducible promoters include responsive elements (REs) which stimulate transcription when their inducing factors are bound. For example, there are REs for serum factors, steroid hormones, retinoic acid and cyclic AMP. Promoters containing a particular RE can be chosen in order to obtain an inducible response, and in some cases, the RE itself may be attached to a different promoter, thereby conferring inducibility to the recombinant gene. Thus, by selecting the appropriate promoter (constitutive versus inducible; strong versus weak), it is possible to control both the existence and level of expression of a therapeutic agent in the genetically modified host cell. If the gene encoding the prophylactic or therapeutic agent is under the control of an inducible promoter, delivery of the agent in situ is triggered by exposing the genetically modified cell in situ to conditions for permitting transcription of the prophylactic or therapeutic agent, e.g., by intraperitoneal injection of specific inducers of the inducible promoters which control transcription of the agent. For example, in situ expression by genetically modified host cells of a therapeutic agent encoded by a gene under the control of the metallothionein promoter, is enhanced contacting the genetically modified cells with a solution containing the appropriate (i.e., inducing) metal ions in situ.

Accordingly, the amount of therapeutic agent that is delivered in situ is regulated by controlling such factors as: (1) the nature of the promoter used to direct transcription of the inserted gene (i.e., whether the promoter is constitutive or inducible, strong or weak); (2) the number of copies of the exogenous gene that are inserted into the host cell; (3) the number of transduced/transfected host cells that are administered (e.g., implanted) to the subject; (4) the size of the implant (e.g., graft or encapsulated expression system); (5) the number of implants; (6) the length of time the transduced/transfected cells or implants are left in place; and (7) the production rate of the biologically active agent or angiogenic factor by the genetically modified host cell. Selection and optimization of these factors for delivery of an effective dose of a particular prophylactic or therapeutic agent is deemed to be within the scope of one of skill in the art, taking into account the above-disclosed factors and the clinical profile of the subject.

The biologically active agent or angiogenic factor can be targeted for delivery to an extracellular, intracellular or membrane location. If it is desirable for the gene product to be secreted from the host cells, the expression vector is designed to include an appropriate secretion signal sequence for secreting the therapeutic gene product from the cell to the extracellular milieu. If it is desirable for the gene product to be retained within the host cell, this secretion signal sequence is omitted. In a similar manner the expression vector can be constructed to include retention signal sequences for anchoring the biologically active agent or angiogenic factor within the host cell plasma membrane. For example, membrane proteins have hydrophobic transmembrane regions that stop translocation of the protein in the membrane and do not allow the protein to be secreted. The construction of an expression vector including signal sequences for targeting a gene product to a particular location is deemed to be within the scope of one of skill in the art.

In an embodiment, vectors for mammalian host cell gene therapy are viruses, more specifically replication-deficient viruses (described in detail below). Exemplary viral vectors are derived from: Harvey Sarcoma virus; ROUS Sarcoma virus; MPSV; Moloney murine leukemia virus; and DNA viruses (e.g., adenovirus) (Temin, Retrovirus vector for gene transfer, in Gene Transfer 149-87 (Kucherlapati, ed., Plenum) (1986)).

Replication-deficient retroviruses are capable of directing synthesis of virion proteins, but are incapable of making infectious particles. Accordingly, these genetically altered retroviral expression vectors have general utility for high-efficiency transduction of genes in cultured cells, and specific utility for use in the method of the present invention. Such retroviruses further have utility for the efficient transduction of genes into host cells in vivo. Retroviruses have been used extensively for transferring genetic material into cells. Standard protocols for producing replication-deficient retroviruses (including the steps of incorporation of exogenous genetic material into a plasmid, transfection of a packaging cell line with plasmid, production of recombinant retroviruses by the packaging cell line, collection of viral particles from tissue culture media, and infection of the target cells with the viral particles) are provided in Kriegler, Gene Transfer and Expression, A. Laboratory Manual (W. H. Freeman Co,) (1990) and Murray, E. J., ed., Methods in Molecular Biology, Vol. 7 (Humana Press Inc.) (1991).

The major advantage of using retroviruses for gene therapy is that the viruses insert the gene encoding the therapeutic agent into the host cell genome, thereby permitting the exogenous genetic material to be passed on to the progeny of the cell when it divides. In addition, gene promoter sequences in the LTR region have been reported to enhance expression of an inserted codling sequence in a variety of cell types (see e.g., Hilberg et al., 84 Proc. Natl. Acad. Sci. USA 5232-6 (1987); Holland et al., 84 Proc. Natl. Acad. Sci. USA 8662-6(1987); Valerio et al., 84 Gene 419-27 (1989)). In vivo gene therapy using replication-deficient retroviral vectors to deliver a therapeutically effective amount of a therapeutic agent can be efficacious if the efficiency of transduction is high and/or the number of target cells available for transduction is high.

Yet another viral candidate useful as an expression vector for transformation of mammalian host cells is the adenovirus, a double-stranded DNA virus. The adenovirus is frequently responsible for respiratory tract infections in humans and thus appears to have an avidity for the epithelium of the respiratory tract (Straus, The Aenovirus 451-96 (H. S. Ginsberg, ed., Plenum Press) (1984)). Moreover, the adenovirus is infective in a wide range of cell types, including, for example, muscle and epithelial cells (Larrick et al., Gene Therapy, Application of Molecular Biology 71-104 (Elsevier Science Publishing Co., Inc.) (1991)). The adenovirus also has been used as an expression vector in muscle cells in vivo (Quantin et al., 89 Proc. Natl. Acad. Sci. USA 2581-4 (1992)).

Like the retrovirus, the adenovirus genome is adaptable for use as an expression vector for gene therapy, i.e., by removing the genetic information that controls production of the virus itself (Rosenfeld et al., 252 Science 431-4 (1991)). Because the adenovirus functions in an extrachromosomal fashion, the recombinant adenovirus does not have the theoretical problem of insertional mutagenesis.

Thus, as will be apparent to one skilled in the art, a variety of suitable viral expression vectors are available for transferring exogenous genetic material into mammalian host cells. The selection of an appropriate expression vector to express a biologically active agent of angiogenic factor amenable to gene replacement therapy and the optimization of the conditions for insertion of the selected expression vector into the cell are within the scope of one of skill in the art without the need for undue experimentation.

In an alternative embodiment, the expression vector is in the form of a plasmid, which is transferred into the target host cells by one of a variety of methods: physical (e.g., microinjection (Capecchi, 22 Cell 479-88 (1980)); electroporation (Andreason et al. 6 Biotechniques 650-60 (1988)); scrape loading, microparticle bombardment (Johniton, 346 Nature 776-7 (1990)); and cellular uptake as a chemical complex (e.g., calcium or strontium co-precipitation, complexation with lipid, complexation with ligand) (Gene Transfer and Expression Protocols in Methods In Molecular Biology, Vol. 7 (E. J. Murray, ed., Humana Press) (1991)). Several commercial products are available for cationic liposome complexation including Lipofectin (Life Technologies, Inc., Gaithersburg, Md.) (Felgner et al., 84 Proc. Natl. Acad. Sci. USA 7413-7 (1987)) and Transfectam™ (ProMega. Madison, Wis.) (Behr et al., 86 Proc. Natl. Acad. Sci. USA 6982-6 (1989); Loeffler et al., 54 J. Neurochem. 1812-5 (1990)). However, the efficiency of transfection by these methods is highly dependent on the nature of the target cell and accordingly, the conditions for optimal transfection of nucleic acids into host cells using the above-mentioned procedures must be optimized. Such optimization is within the scope of one of skill in the art.

In an embodiment, the preparation of genetically modified host cells contains an amount of cells sufficient to deliver a biologically active agent or angiogenic factor effective dose of a disrupted gene of the present invention to the recipient in situ. The determination of an effective dose of the biologically active agent or angiogenic factor is within the scope of one of skill in the art. Thus, in determining the effective dose, the skilled artisan would consider the condition of the patient, the severity of the condition, as well as the results of clinical studies of the biologically active agent or angiogenic factor being administered.

The invention embraces a tissue-engineered cardiac construct graft. The graft may comprise a plurality of the above-described genetically modified cells attached to a support that is suitable for implantation into a mammalian recipient in accordance with standard surgical procedures, specifically into the heart. The support can be formed of any natural or synthetic material described above.

Polypeptides

The invention also involves the use of polypeptides, which may enhance the properties of the tissue-engineered construct during in vitro culturing and following implantation, such as the ability of endogenous cells to populate the construct, to enhance the growth of seeded cells, to enhance migration, to enhance function, to facilitate angiogenesis, to reduce infection, to reduce the likelihood of thrombus formation and so on. Polypeptides thus may include soluble peptides, Ig-tailed fusion peptides, members of random peptide libraries (see, e.g., Lam et al., 354 Nature 82-4 (1991); Houghten et al., 354 Nature 84-6 (1991)), combinatorial chemistry-derived molecular library made of D- and/or L-configuration amino acids, and phosphopeptides (including members of random or partially degenerate, directed phosphopeptide libraries, see, e.g., Songyang et al., 72 Cell 767-78(1993)).

Such polypeptides may include those derived from the transcription and translation of the gene encoding the biologically active agent or angiogenic factor. The term analog refers to any polypeptide having an amino acid sequence, in comparison to the amino acid sequences of the polypeptides of the present invention, in which one or more amino acids have been substituted with other amino acids, where the substituted amino acids allow or require the polypeptide to assume the equilibrium conformation of the domain of the parent protein. Often, cysteine, lysine and glutamic acid will be used for their side chains which can form covalent linkages to restrict the conformation of a peptide.

The polypeptides of the invention may be a naturally purified product, or a product of chemical synthetic-procedures, or produced by recombinant techniques from a prokaryotic or eukaryotic host (for example, by bacterial, yeast, higher plant, insect and mammalian cells in culture). Depending upon the host employed in a recombinant production procedure, the polypeptides of the present invention may be glycosylated with mammalian or other eulkaryotic carbohydrates or may be non-glycosylated. Polypeptides of the invention may also include an initial methionine amino acid residue.

In one embodiment, the one or more polypeptides of the invention may be added to the bioreactor culture media to enhance the properties of the tissue-engineered construct during in vitro culturing, such as to enhance the growth of seceded cells, to enhance migration, to enhance function, to facilitate angiogenesis and so on. In a specific embodiment the polypeptide to be added to the bioreactor media includes an angiogenic factor and more particularly, a polypeptide encoding VEGF, to promote angiogenesis and to produce a vascularized 3D tissue-engineered cardiac construct.

An effective amount of VEGF is added to the bioreactor cell culture at an amount in the range of about 1 ng/ml to about 500 ng/ml and the cells are incubated at 37° C. in an atmosphere of about 95% humidity to about 100% humidity with CO₂. The media has a final pH of 7.2. The cells are assessed for vessel formation by either microscopy or by placing a VueLife bag directly on the microscope stage and the culture media is changed about every 48 hours. The selection of conditions suitable for cell growth, including optimization of the effective amount of the angiogenic factor, such as VEGF, to be added to the bioreactor cell culture to promote angiogenesis and produce a vascularized 3D tissue engineered cardiac construct are within the scope of one of skill in the art without the need for undue experimentation.

Alternatively, the polypeptide encoding the biologically active agent or angiogenic factor may be present in a pharmaceutical composition in admixture with a pharmaceutically acceptable sterile vehicle. The pharmaceutical composition may be compounded according to conventional pharmaceutical formation techniques. For example, the vehicle will usually comprise sterile water, although other ingredients to aid solubility or for preservation purposes may be included. The pharmaceutical composition may be coated onto the vascularized 3D tissue-engineered construct prior to implantation in the subject in accordance with standard surgical procedures. The effective amount of the polypeptide to be administered will depend on the activity of the particular biologically active agent or angiogenic factor administered, which may readily be determined by those of ordinary skill in the art.

Without further elaboration, it is believed that one skilled in the art, using the preceding description, can utilize the invention to the fullest extent. The following examples are illustrative only, and not limiting of the disclosure in any way whatsoever.

### EXAMPLES

### Specific Example 1

### Preparation of Tissue Engineered Cardiac Constructs Preparation of Neonatal Rat Cardiac Cells (NNRCC)

This example describes the preparation of a cardiac engineered construct using a bioreactor system. Heart were dissected using sterile technique from neonatel (2 day old) Sprague Dawley rats (Hilltop Farms, Scottdale, Pa.). Cells were prepared using the Neonatal Cardiomyocyte Isolation System (Worthington Biochemical Corp., Freehold, N.J.). Bioreactor (HARV) and standard tissue culture plates were run in parallel and assessed.

Briefly, the isolation system was performed in the following manner. 100 mm culture dishes were filled with about 25 ml of sterile Hanks Balanced Salt Solution (HBSS) and fragments of tissue were placed into the culture dish using sterile techniques. Each fragment was examined and any pulmonary or other non-cardiac tissue that was present was removed.

This tissue was then transferred to a clean, dry 100 mm dish and minced into about 1 mm² to about 2 mm² pieces. The minced tissue was transferred to a water-jacked spinner flask. After the minced tissue was placed in the flask, the remainder of the sterile HBBS was added to the tissue to rinse away excess blood cells and debris; the rinse solution was then decanted leaving the tissue fragments in the flask. Proteolytic enzyme solution was added to the minced tissue at a ratio of about 2:1 (i.e., about 2 volumes of enzyme per volume of tissue). The flask was equipped with a stirrer and positioned on a magnetic stir plate with the rite of revolution set to just suspend the fragments, specifically about 200 rpm. The solution and tissue were incubated for about 15 minutes. Then, the enzyme solution was decanted into a waste beaker. This step was repeated 1 time. The solution was stirred to suspend the fragments and incubated for about 20 minutes.

After incubation, the enzyme solution was decanted into a 50 ml centrifuge tube containing about 15 ml of growth medium. The cells were collected by gentle centrifugation at about 250 x g for about 10 minutes. Following centrifugation, the solution was decanted from the pellet into the waste beaker with care not to dislodge the soft pellet. The pellet was resuspended in about 5 ml of growth medium. This step as repeated until the enzyme solution was clear after about 20 minutes incubation.

The collected cells were pooled and gently triturated to resuspend the cell suspension. Cell isolates consisting of less than about 70% myocardial cells as estimated by myosin heavy-chain staining were excluded. The cells were diluted with a defined, serum-free heart medium (SFHM) at a density of 1×10⁶ cells/ml.

Preparation of Cell Supports

The support material including oriented fibers of type I collagen (Organogenesis Inc., Canton, Mass.) was rinsed in sterile, deionized water and stored in sterile Dulbecco's Phosphate Buffered Saline Solution. A stock solution of fibronectin (Collaborative Research, Waltham, Mass.) was prepared by adding about 10 ml of sterile deionized water to a 1 mg vial of fibronectin. Alternatively, a laminin stock solution may be used and prepared in the same manner. The support material was then placed into the fiberonectin stock solution for about 24 hours at about 4° C. After the incubation period the support material rinsed in cell-culture medium just prior to use.

HARV-Bioreactor Cell Culture

Cells were maintained in a SFHM at a density of 1×10⁶ Trypan-Blue (Sigma Chemical Co., St. Loius, Mo.) excluding cells per ml of medium, for every 4.8 cm² of culture surface area. Cells were allowed to adhere for about 24 hours and were then fed every 48 hours for the duration of the experiments. Low-shear suspension culture was performed using NASA-designated HARV bioreactors (Synthecon Inc., Friendwood, Tex.).

The bioreactor was thoroughly rinsed with deionized water taking care to assure that foreign material was not introduced and no surfactants were used. The bioreactor was sterilized by autoclaving. Alternatively, the bioreactor may be sterilized by rinsing with 70% ethanol. The bioreactor was rinsed again in sterile, deionized water and loaded with the prepared support, NNRCC suspension at a density of about 1×10⁶ cells/m;, and SFHM.

The bioreactor was attached to a rotating platform having a variable speed motor of about 0 rpm to about 100 rpm and a simple mounting platform. The rotation rate of the bioreactor was set to keep the support material and cells in static suspension. To suspend cells and allow 3D associations, HARV bioreactors and Teflon®-bags were slowly rotated (at about 30 RPM) around an axis orthoganol to the gravity vector. The clinostatic rotation of cultures and the resultant decrease in gravitational influences allows cells to be quiescently suspended without the introduction of high levels of damaging fluid shear. Such a low shear environment is especially suitable for cardiac cells, which are known to be exquisitely sensitive to mechanical stimulation.

As the bioreactor rotated, it was possible to visualize the elliptical path traced by the support surfaces using oblique illumination from a penlight. As the rate of rotation was increased, the elliptical paths got smaller until the support appeared to remain stationary relative to the wall of the bioreactor. The bioreactor was placed into an incubator at 37° C. in about 95% to about 100% humidity CO₂. The final pH of the medium was about 7.2.

The NNRCC culture was periodically assessed to determine the extent of attachment of the cells to the support material and to observe the contractile ability (i.e., beating) of the attached cells. The bioreactor culture was assessed by either microscopy or by placing a VueLife bag (American Fluoroseal Corp., Gaithersburg, Md.) directly on the microscope stage. The results showed that cells attached to the support materials within about 48 hours and were spontaneously beating within about 72 hours. The SFHM was changed in about 48 hour intervals.

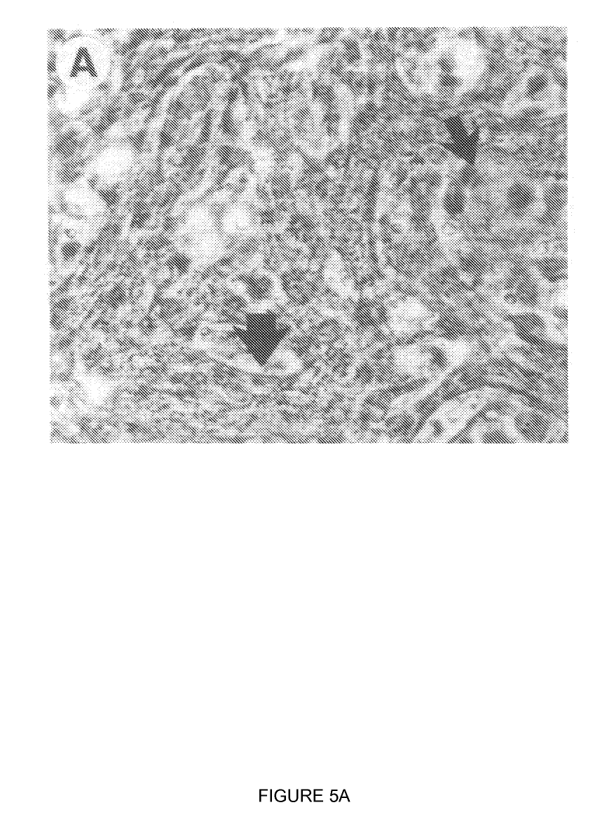

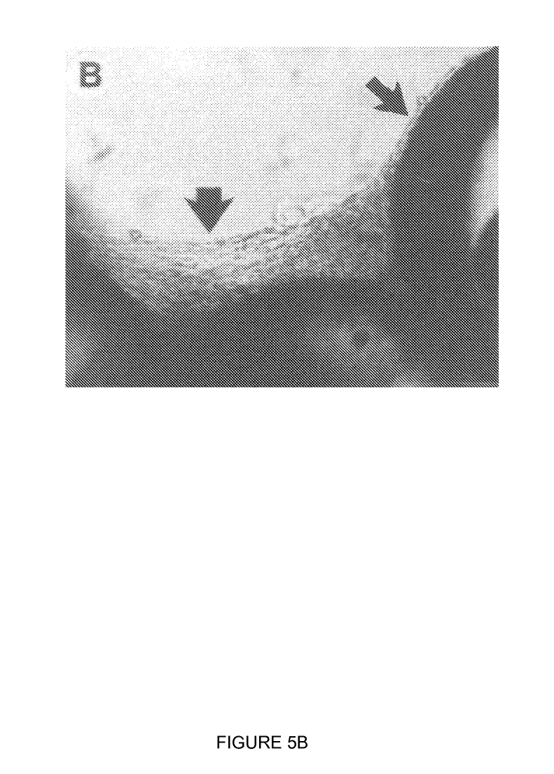

The NNRCCs cultured in the HARV-bioreactors formed complex aggregates along and between the included support scaffolds. Cells cultured on fibronectin-coated polystyrene spheres developed architectures that differed substantially from the general organization of cells seen in parallel culture that were provided the same cell attachment substrate. As seen in FIGS. 5A-5B, bioreactor-cultured cells organized into 3D aggregates as opposed to the two-dimensional (2D) mosaic pattern seen in traditional cultures. The overall size of HARV-derived 3D aggregates depended on the total biological load within the bioreactors, but when the ratio of volume-of-medium to scaffold-surface-area to cell number was the same as that used in traditional culture method (i.e., 1 ml medium/4.8 cm² of fibronectin-coated polystyrene/1×10⁶ cells), aggregate sized averaged between about 6 to about 8 beads per cluster. Clusters ranged in size along a Poisson distribution (not shown) with grouping of up to about 25 beads occasionally found. Necrosis as assessed by microscopic and histologic examination, was not evident in the bioreactor cultures, even within the largest clusters.

Cells cultured in 3D-aggregates were metabolically very similar to cells cultured in 2D-monolayers by traditional methods. As shown in Table 1 below specific activities for creatine kinase (CK), glucose-6-phosphate dehydrogenase (G-6-PDH), 6-phosphogluconate dehydrogenase (6-PGDH) hexokinase (HK), isocitrate dehydrogenase (ICD), malate dehydrogenase (MDH), NAD-dependent cytochrome c-reductase (NCR), and pyruvate kinase (PK) were the same in HARV-derived cultures as in monolayer cultures derived by standard methods.

TABLE 1
Metabolic Activity in Standard and HARV-based Cultures

| | Specific Activity | Specific Activity |
|---|---|---|
| Enzyme | (Standard Culture) | (HARV culture |
| CK | 669.8 ± 18.84 | 622.6 ± 24.13 |
| 6-PGDH | 21.57 ± 2.205 | 22.71 ± 2.361 |
| G-P-PDH | 25.66 ± 1.761 | 26.45 ± 1.324 |
| HK | 54.20 ± 7.282 | 42.68 ± 3.012 |
| ICDH | 32.82 ± 1.571 | 28.20 ± 2.143 |
| MDH | 125.3 ± 13.64 | 124.1 ± 14.05 |
| NCR | 23,050 ± 2675 | 24,050 ± 2910 |
| PK | 355.3 ± 49.89 | 328.1 ± 39.71 |

The specific activities in Table 1, above, were estimated from unit activities divided by total protein concentrations and were given as the mean±SEM; n=6 experiments. The samples were collected after 6 day of culture and stored at −70° C. until assayed. The data are presented as the number of units (nmoles of NAD, NADP, or cytochrome c reduced per minute) per mg of protein assayed. No significant differences were found between standard and HARV cultures by Student's t test with Bonferroni correction (p=0.05). These results suggest that the metabolic function of the cells did not vary with culture type. In additional the results indicated that HARV-cultures cells received adequate nutrition and that the accumulation of waste products was not a problem in the bioreactors.

The 2D and 3D systems were further characterized by evaluating the cellular composition of the initial cultures. To estimate the percentage of myocytes within the cultures, cells were dissociated after 72 hours in culture, collected and immediately fixed. Samples were stained with Hoechst 3358 (to label all nuclei) and anti-MyHC (to label myocytes), placed onto microscope slides and enumerated. The number of nuclei within MyHC-positive cells was compared with the total number of nuclei in randomly selected microscope fields to determine the proportion of cardiomyocytes in HARV-derived/3D and standard/2D cultures. The percentage of myonuclei ranged from about 70% to about 80% depending on the particular cell isolation, and there were no statistically significant differences found between culture methods (75±4% for 2D and 75±3% for 3D; mean±SD, n=five cell isolations). In addition, the proportion of bi-nucleated myocytes was not significantly different in 3D and 2D cultures (32±3% binucleated for 3D and 29±4% for 2D; mean±SD, n=4). Moreover, the total amount of DNA was directly related to the amount of protein in these samples and did not vary between 2D and 3D systems (0.352±0.012 μg-DNA/mg-protein for 2D and 0.341±0.015 μg-DNA/mg-protein; mean±SD, n=25 for 2D and n=12 for 3D) suggesting that the hypertrophic index of the cells was the same in both types of culture. In conjunction with the metabolic data in Table 1, these observations indicated that HARV culture system, the traditional culture system, and the tissue were very similar in terms of general cellular content. Most notably, the proportion of myocytes in intact neonatal rat ventricles is approximately 75% with the remaining of the cell population being dominated by endocardial cells and fibroblasts. This ratio, which is important in the tissue engineering of cardiac constructs, was maintained in these HARV-cultures in this example.

To specifically evaluate the initial condition of the cardiomyocytes within HARV-based and traditional cultures, the levels of MyHC and f-actin were quantified using fluorescent probes. Cultures were allowed to progress for about 72 hours after initial seeding. Samples were then collected and fixed. The level of MyHC-related fluorescence was normalized to nucleic acid fluorescence and the resulting value was used as an estimation of myocyte-specific gene expression. The ratio of f-actin fluorescence to nucleic acid fluorescence was used similarly as an estimate of sacromere assembly and hypertrophic index within the same myocytes. No significant differences were found between the bioreactor and non-bioreactor cultures. The fluorescence quantization indicated that the functional condition of the myocytes was nearly identical in HARV-based and standard cultures and that, overall, the cells comprising each type of in vitro system were qualitatively the same.

### Specific Example 2

### Contractile Function of In Vitro Cardiac Constructs

Primary NNRCCs in SFHM form spontaneously and rhythmically contracting cultures within the first twenty hours after plating in monolayer cultures. To directly assess contractility and the modulation of contractile activity by adrenergic cells, the effects of propranolol, which is a beta-blocker on the frequency of spontaneous contraction in 2D and 3D preparation were evaluated. 3D cardiac cell cultures were prepared in transparent Teflon® culture bags that were slowly rotated to quiescently suspend the cells and HFN-coated polystyrene beads. Cultures were observed on a 37° C. microscope stage and the spontaneous contractile frequency of the constructs was determined. Samples were then exposed to a pharmacological dose of propranolol without disturbing the field of view on the microscope stage. Three minutes later, the contractile frequency was reassessed. Results are summarized in Table 2 below.

TABLE 2
Effect of Adrenergic Agents of Contractile Activity

| | | Baseline | |
|---|---|---|---|
| | | beat frequency | |
| | Baseline beat | (Propranolol | Postproprandolol |
| Sample | Frequency (all) | responders) | beat frequency |
| 2D/culture | 195.59 (±7.472) | 189.15 (±13.54) | 130.86 (±10.23) |
| dishes | (n = 25) | (19 of 25) | 30% decrease |
| 3D/HARVs | 47.78 (±4.766) | 52.42 (±5.332) | 34.59 (±3.842) |
| | (n = 12) | (6 of 12) | 34% decrease |

As expected, both culture types were spontaneously contractile. In standard (2D) preparations entire culture wells beat in unison. In the 3D constructs, entire aggregates of cells in physical contact beat in unison. The baseline rate on contraction, however, was significantly lower in the 3D aggregates than in the parallel 2D culture wells (Student's t test, p<0.05); the reason for this difference may be related to the presence of low levels of fluid shear found in the HARV bioreactors. Despite the apparent difference in baseline contractile rate, cells in both types of culture responded to the adrenergic antagonist propranolol in a similar manner. Propranolol caused a decrease in beat frequency in a subset of cultures in both 3D and 2D conformations. These data suggest that the adrenergic cells present in the rodent heart were maintained and that they re-established functional interactions with the cardiomyocytes in vitro.

### Specific Example 3

### Assessment of Cultures by Scanning Electron Microscopy

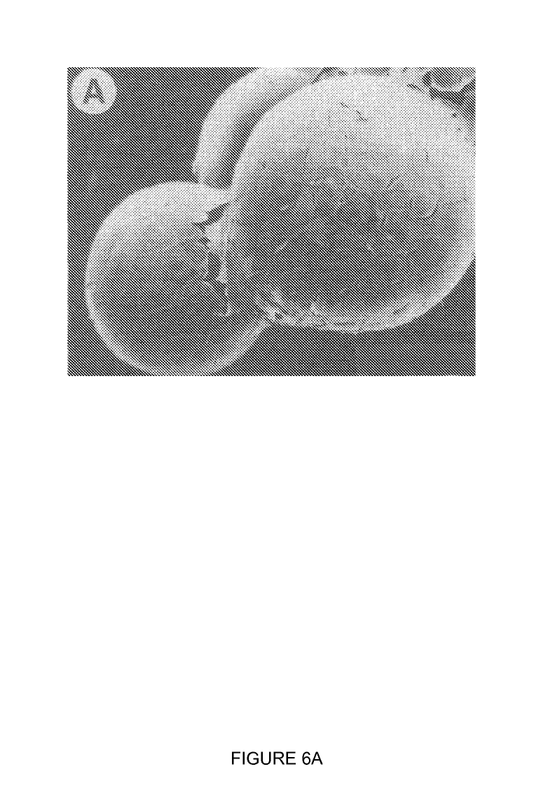

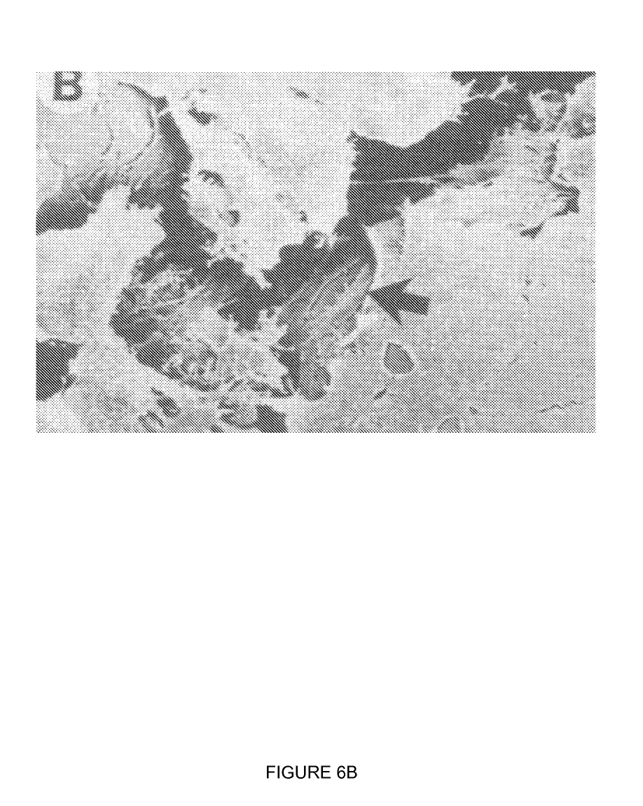

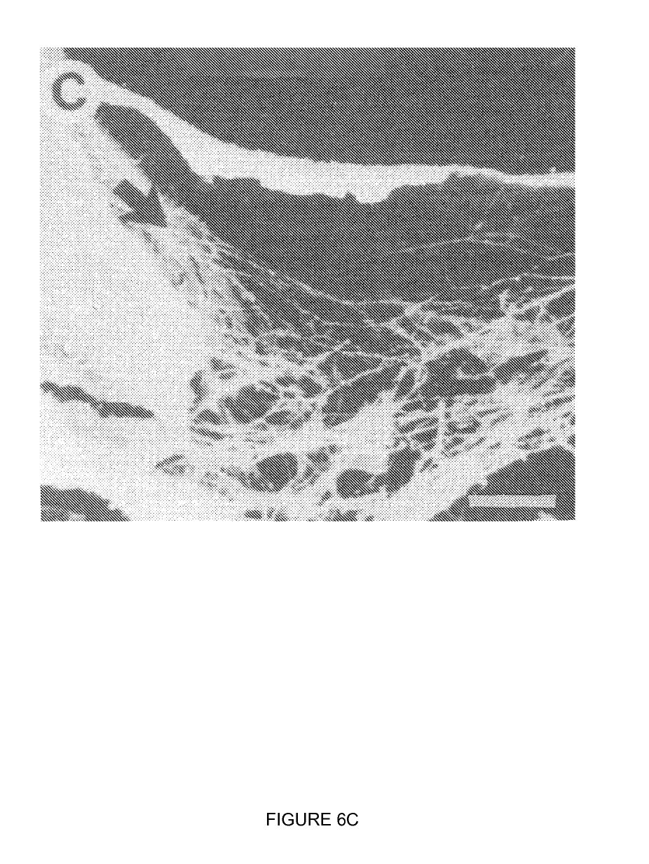

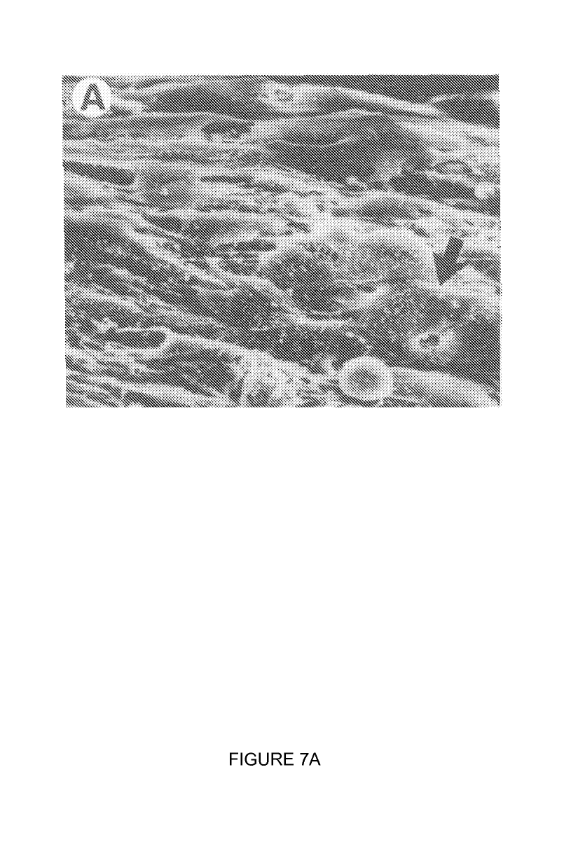

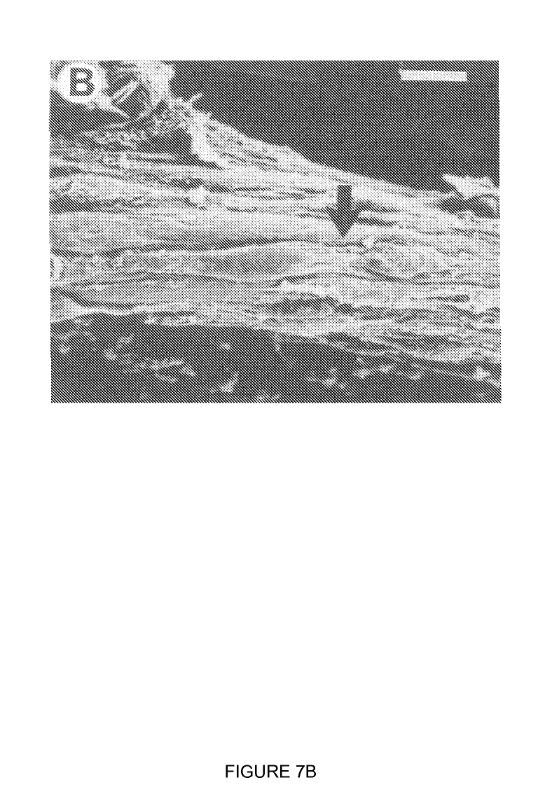

To discern the cellular organization present in HARV-derived cultures, the morphology 3D cultures were evaluated using scanning electron microscopy (SEM). Three morphological classes of cells are typically ascribed to populations of cardiac cells: elongate myocardial cells, cuboidal endocardial cells and fibroblast-like cells. In standard tissue culture dishes, the influence of gravity caused the formation of thin monolayers of these cells into a less organized, mosaic-like monolayer; however, as shown in FIGS. 6A-6B, cells were differentially distributed by apparent functional class within the 3D structures generated by HARV-bioreactor culture. This differential distribution into layers of cells arose during the first 6 days of culture, was maintained through subsequent culture periods, and was accompanied by the production of extracellular matrix material within the aggregates as shown in FIG. 7C. Most notably, the distribution of cell types in 3D cultures recapitulated the distribution of cells seen in vivo. A thin layer of endocardial-like cells formed a barrier between the medium and a layer of substrate-associated myocardial cells. The distribution of cell layers was similar to the organization of the tissue from which the cells were originally isolated with the outside of the aggregates corresponding to the inside of the heart.

Threads of oriented collagen, made up of protein filaments running parallel to the long axis of the thread, were used in place of HFN-coated polystyrene beads in some cultures. The collagen threads provided an oriented substructure along which the cells could orient. As shown in FIGS. 7A-7B, neonatal cardiac cells grown in 3D aggregates on collagen fibers formed elegant networks of aligned cells. Multiple cell layers were visible on single fibers and occasionally between neighboring fibers. Longitudinally apposed myocyte-like cells formed interdigitating structures, which were reminiscent of myocardial cell associations seen in vivo. Overall, the SEM findings were consistent with the establishment of an outer layer of squamous epithelium around an inner layer of elongated cells in HARV-based culture. This arrangement closely resembled architecture of the heart in vivo.

### Specific Example 4

### Assessment of Cultures by Transmission Electron Microscopy (TEM)

To further delineate the organization and distribution of cells within the HARV-generated constructs, and to examine the ultrastructure of the cells within 3D constructs, transmission electron microscopy (TEM) was carried out on collagen fiber-based cultures similar to those shown in FIGS. 7A-7B. Fibers were sectioned down the tong axis o the collagen thread to expose the longitudinal organization. Morphological identification of the major cell types was possible in the TEM because cardiomyocytes contain distinctive sarcomeric structures, adrenergic cells possess distinctive ganules, and endothelial cells are heavily vesiculated with fenestrations and a distinctive cytosolic appearance.

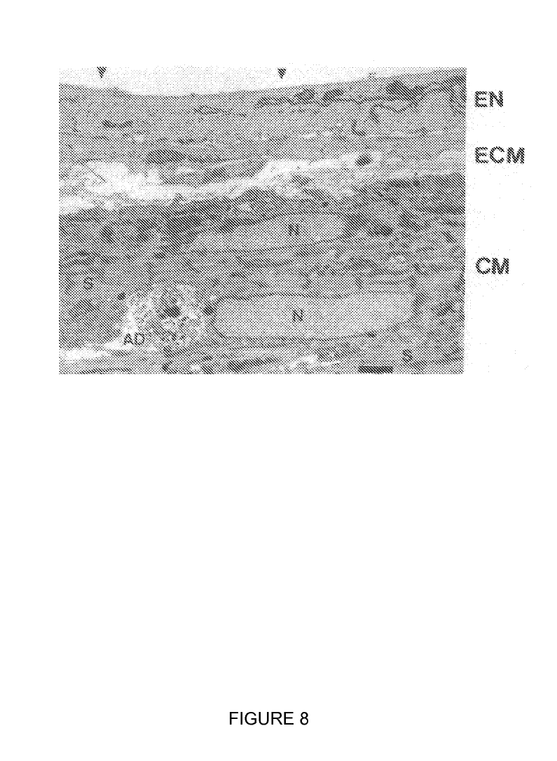

As shown in FIG. 8, a tissue-like organization of cells was evident in the constructs. Myocytes were arranged in register near the thread surface, which corresponded to the interior of the ventricular myocardium. Moving outward from the myocytes, an extracellular matrix layer, resembling the subendocardial layer of tissue, was present within the constructs. Finally, a thin endothelial cell layer was found at the outer surface of the aggregates where the constructs contacted the medium. Not surprisingly, the myocytes in direct contact with the collagen surface were oriented to the axis of the thread; however, the large number of myocytes that were not in contact with the collagen were also oriented parallel to the thread axis. The means by which the alignment of the outer myocytes to the collagen was accomplished is unclear, but the organization was likely mediated by the underlying myocytes. Overall, the layered structure observed in the TEM verified the tissue-like distribution of cells suggested by the SEM analysis.

During the TEM observations, cell to cell interactions characteristic of cardiac tissue were observed. These was evidence that structures resembling adrenergic nerve endings had formed. Membrane delimited bodies containing vesicles and granules consistent with adrenergic innervation sites were found within the myocardial cell layer of the constructs. As can be seen in FIG. 8, these bodies likely formed after establishment of the cardiomyocytes because the sarcomeric structure of the adjoining myocytes was deformed in the areas containing them. The appearance of these structures, combined with the effects of adrenergic agents discussed above, suggests that adrenergic cells were present in the primary cell isolate and that these were able to re-establish functional interaction with the surrounding cells. It should be noted that the cholinergic antagonist atropine did not result in an alteration of baseline contractile frequency, and cholinergic bodies were not seen during our TEM analyses.

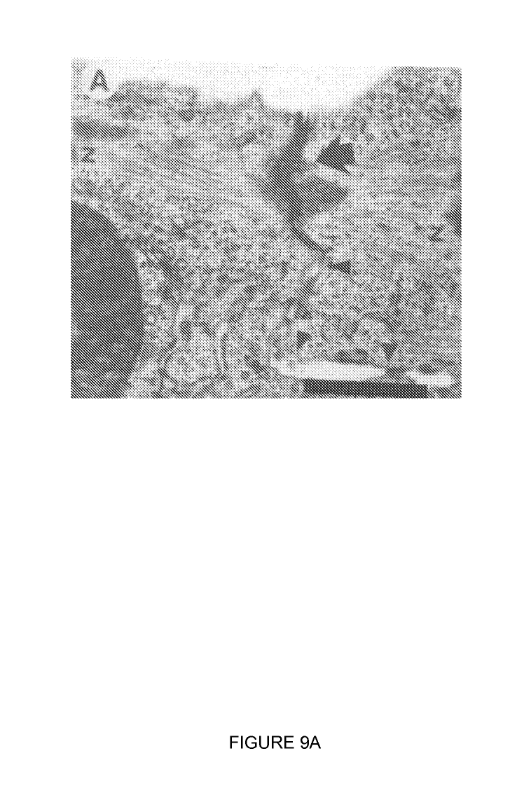

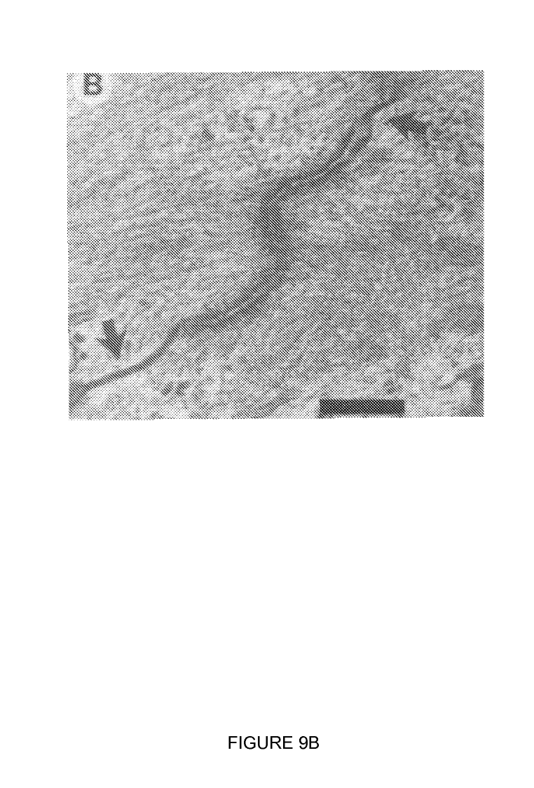

Cellular junctions consistent with mechanical and electrical connections among cardiac cells were also prevalent in our preparations. As shown in FIGS. 9A-9B, fascia adherens junctions associated with mechanical connection among cardiomyocytes, and gap junction associated with the propagation of electrical impulses through the myocardium. These cell junctions are consistent with the appearance of intercalated discs in our SEM preparations. Overall, the ultrastructural organization of the 3D constructs appeared remarkably like that of the intact rat heart.

Interestingly, the appearance of organelles in the HARV-cultured cells was also tissue-like. It has been observed that, in vitro, cardiomyonuclei tend to locate basally near the support surface, and that sarcomeres tend to locate predominantly in apical regions. This is not the organization of organelles seen in the tissue. In HARV-based cultures, a sampling of fifty different myocytes was selected so that both nuclei and sarcomeres were clearly evident in each section. Nuclei were as likely to be located apically (18/50) or centrally (19/50) as they were to be located toward the support surface (13/50). This distribution of organelles was tissue-like, and, combined with the tissue specific junctions and multicellular organization, gave HARV-derived cultures the ultrastructural appearance of tissue.

### Specific Example 5

### Role of BMP-2 and Notch Signaling Pathway in Formation of 3D Tissue-Like Cardiac Construct

In this example, bioreactor (HARV) and standard tissue culture plates were run in parallel and assessed. In this example, hearts were dissected using sterile technique from neonatel (2 day old) Sprague Dawley rats (Hilltop Farms, Scottdale, Pa.). Cells were prepared using the Neonatal Cardiomyocyte Isolation System (Worthington Biochemical Corp., Freehold, N.J.).

Primary Cell Isolation and Culture

2 day old neonatal rat pups were halothane anesthetized and cardioectomitized. The isolated hearts were minced and subjected to about 16 hour digestion by about 0.75 g/ml trypsin, followed by about 0.33g/ml collagenase II digestion and cell straining to completely separate individual cells. The cells were then counted on hemocytometer with trypan blue staining aid resuspended in modified serum-free cardiac media (SM3+) at a density of about 1×10⁶ total cells/ml. Cells were then mixed with fibronectin-coated Nunclon plastic microcarriers at (insert surface area/volume media ration) and infused into PTFE bags for bioreactor rotation, with the remainder plated onto fibronectin-coated six-well Nunclon plastic plates at a density of about 2×10⁶ cells/well as a control population. Feeding was performed at about 24 hours post-inoculation to remove unattached cells, and about every 3 days thereafter. Samples were collected prior to culture start (Day 0) and at varying intervals thereafter. For the Noggin treatment protocol, about. 0.4 μg/ml Noggin (Pepro Tech, London, UK) in about 10 mM acetic acid was added to media just prior to inoculation and to media used for feeding, with control using untreated media.

RNA and Protein Isolation

The samples were collected in TriReagent (MRC, Cincinnati, Ohio) at about 1 ml TriReagent/1×10⁶ cells, plate samples were scraped while bioreactor-cultured samples were isolated by collecting the microcarriers in TriReagent and vortexing. BCL reagent was then added to each sample at about 100 μl/ml and the aqueous phase removed. RNA was precipitated from the removed aqueous phase using ethanol and subjected to RNAse-free DNAse digestion using a DNA-free™ kit (Ambion, Austin, Tex.) The remaining TriReagent then had the total protein from the isolation precipitated via isopropanol and resuspended in LDS solution (Invitrogen, Carlsbad, Calif.) with added pepstatin and leupeptin.

Reverse Transcriptase and Real-Time PCR

About 1 μg isolated RNA was converted to cDNA via 20 μl reaction with reverse transcriptase (Ambion, Austin, Tex.) reaction using oligo dT primers. Resulting cDNA was then quantified via spectrometer and diluted to about 0.25 pg/ml each sample. Real-time PCR amplification was then performed with each sample loaded with about 50 ng/reaction and plated in triplicate; real-time amplification was performed using both ABI Prism 6700 and BioRad MyiQ real-time PCR machines using ABI SYBR Green Master Mix and BioRad SYBR Green kits, respectively. The PCR primers used were as follows:
BMP2: 5′-tgaacaca gctggtctcagg and 3′-gctgtttgtgtttggcttga;
Notch 1: 5′-tgttgtgctcctgaagaacg and 3′-gcaacactttggcagtgtca;
Notch 2: 5′-tggaggcttcacctgtctct and 3′-cagacactggaagcgattga; and
Jagged 1: 5′-gtcccactggtttctctgga and 3′-ettgccctcgtagtcctcag.
   Experimental sample cT values were then normalized to cT values for Troponin 1 at same time point, then fold difference from value at Day 0 time point was determined.

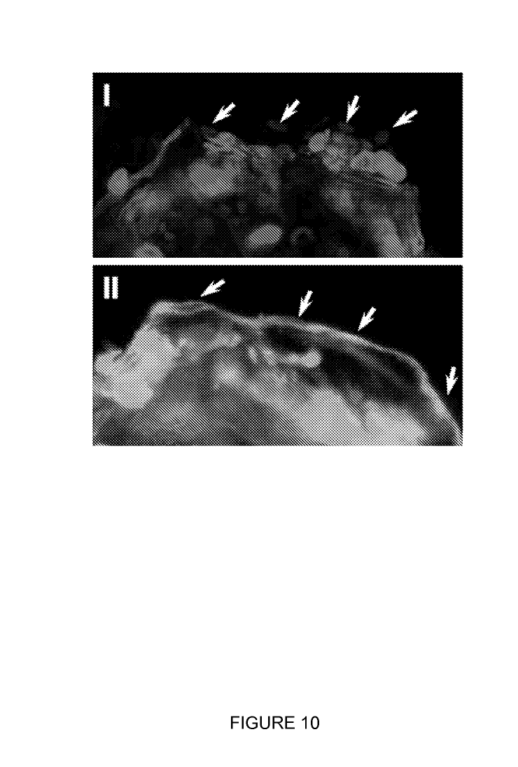

Examination of the bioreactor tissue engineered cardiac constructs using immunofluorescent staining against myosin for myocyte localization and vimentin for non-muscle cells, showed changes in their relative positions over time. While initially endothelial cells and myocytes showed a randomized distribution in their binding to microcarrier surfaces, over time a redistribution of the cells was observed, with endothelial cells covering the attached myocytes in the resulting tissue-like aggregates (FIGS. 6A and 10). Myocyte cell beating was observed as early as about 8 hours post-inoculation, which may suggest a relatively rapid association of the bioreactor cultured myocytes to each other and reassembly of the contractile apparatus.

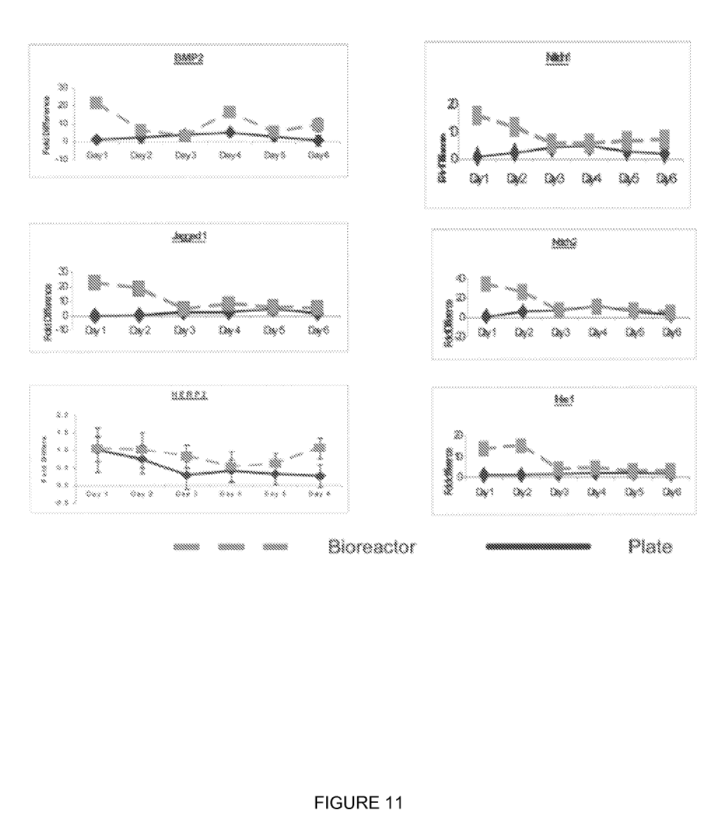

Real-time PCR of the genes suspected in mediating cellular rearrangement showed marked differences in mRNA transcript levels in bioreactor-cultured cells as compared to plate-growth cells (FIG. 11). In each case, mRNA transcript levels showed a heightened level at about 24 and about 48 hours post-inoculation in the HARV bioreactor cultures. This pattern was relatively constant in BMP2 and the Notch 1/Notch 2 receptors, as well as the Notch-binding ligand Jagged 1. This pattern was also visible in the transcript level of Hes1, a known downstream target of Notch signaling, which may suggest an increase not only in the levels of Notch receptor but an increase in the level of effective Notch signaling occurring within HARV bioreactor culture. Surprisingly, the Notch downstream target HERP2 did not show similar increases in mRNA transcript, suggesting a particular pattern of Notch-mediated downstream signaling may be active in the HARV bioreactor cell population.

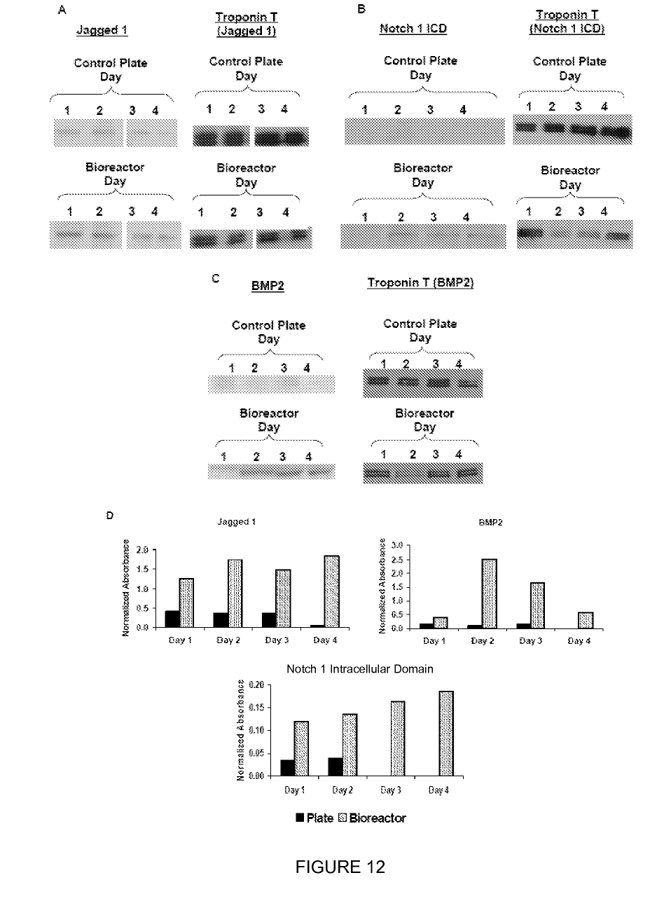

Further evidence for changes in gene activity were examined using Western blot (FIG. 12). Consistent with the observed increase in mRNA levels, protein levels also showed a heightened level for the proteins of interest. In particular, BMP 2 protein levels showed substantial increases over the low levels observed in the control 2D plate cultures. Furthermore, Jagged 1 also showed a substantial increase over the low levels of Jagged 1 observed in the control 2D plate cultures. The antibody used for the Notch 1 protein analysis was specific to the N-terminal section of the Notch 1 intracellular domain (ICD). Importantly, this epitope is only exposed when the Notch 1 receptor is cleaved by γ-secretase; therefore, the high levels of this activated form of Notch 1 detected in HARV bioreactor cardiac constructs suggested an increase in active Notch signaling.

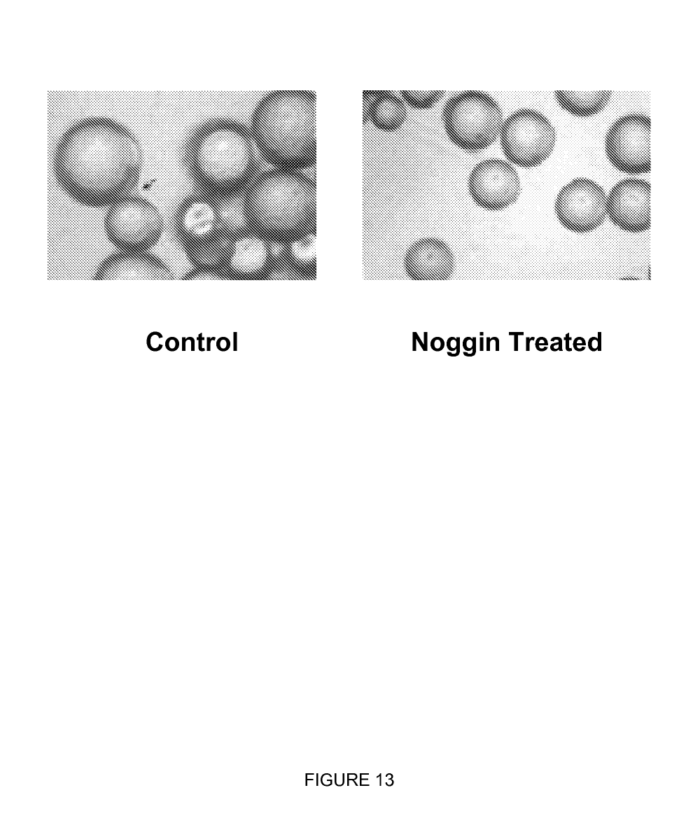

Moreover, Noggin was used to assess the role of BMP2 in the formation of tissue-like aggregates in bioreactors. Noggin is a secreted factor which acts as an antagonist for members of the BMP family of proteins (i.e., in solution, Noggin will bind BMP 2 and prevent BMP 2 from binding to its receptor). Thus, adding an effective amount of Noggin to the culture media may suppress BMP 2 function. As shown in FIG. 13, the addition of Noggin to the culture media facilitated the disruption of the tissue-like aggregate structure. The control bioreactor cardiac cultures formed large tissue-like aggregates surrounding multiple microcarriers complete with substantial thick structure formation visible between the microcarriers. In direct contrast, cultures treated with Noggin had only small thin aggregates in the range of about 2 microcarriers to about 3 microcarriers or no tissue-like aggregate formation at all. Since no damage consistent with disruption of larger aggregates was observed, these results indicated that the reduction in aggregate size was from the inhibition of initial aggregate formation rather than the breakdown of standard large aggregates. Also, contractile activity was observed in about 8 hour post-inoculation Noggin-treated cultures as standard, suggesting no disruption in myocyte interaction or contractile apparatus reformation was occurring as a result of the addition of Noggin to the culture media.

The examples given above are merely illustrative and are not meant to be an exhaustive list of all possible embodiments, applications or modifications of the invention. Thus, various modifications and variations of the described methods and systems of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention which are obvious to those skilled in the cellular and molecular biology fields or related fields are intended to be within the scope of the appended claims.

The disclosures of all references and publications cited above are expressly incorporated by reference in their entireties to the same extent as if each were incorporated by reference individually.

What is claimed is:

1. A method for producing a three-dimensional vascularized cardiac construct, said method comprising the steps of:
culturing cardiac cells in a bioreactor vessel containing a support under appropriate conditions to facilitate cell growth on the support;
adding an effective amount of a first biological active agent to the cardiac cell culture to facilitate cardiac cell migration, differentiation and organization into a three-dimensional construct; and
adding an effective amount of a second biological active agent to the three-dimensional cardiac cell construct to promote vascularization of the three-dimensional cardiac construct.

2. The method of claim 1, wherein the cardiac cells are one or more mammalian cells selected from the group consisting of cardiomyocytes, endocardial cells, cardiac adrenergic cells, cardiac fibroblasts, vascular endothelial cells, smooth muscle cells, cardiac progenitor cells, and stem cells.

3. The methods of claim 1, wherein the appropriate culturing conditions in said culturing step includes culturing the cardiac cells in the presence of a serum-free media.

4. The method of claim 1, wherein the support is one or more materials selected from the group consisting of sutures, meshes, foams, gels, ceramics, acellularized extra-cellular matrix material.

5. The method of claim 4, wherein the structure fabricated from one or more components selected from the group consisting of silk, polypropylene, polyamide, polyvinylidene, polyester, polyether, polydioxanone, nylon, linen, cotton, plain gut, chromic gut, poliglecaprone, polyglactin, polylactide, collagen, or naturally occurring protein, and any combination thereof.

6. The method claim 1, wherein the first biological active agent in said first adding step is one or more compounds selected from the group consisting of bone morphogenetic protein (BMP), noggin, notch, notch ligand, modulators of BMP signals, and modulators of BMP signals.

7. The method of claim 1, wherein the second biological active agent in said second adding step is one or more compounds selected from the group consisting of FGF, bFGF, acid FGF (aFGF), FGF-2, FGF-4, EGF, PDGF, TGF-betal, angiopoietin-1, angiopoietin-2, PlGF, VEGF, and any combination thereof.

8. A three-dimensional cardiac construct having similar physiological characteristics of intact in vivo cardiac tissue produced by the method of claim 1.

9. A method for treating a subject afflicted with cardiac damage, said method comprising the steps of:
obtaining a three-dimensional vascularized cardiac construct organized on a support produced by the method of claim 1; and
implanting the three-dimensional vascularized cardiac construct in the subject.

10. The method of claim 9, further comprising the step treating the three-dimensional cardiac construct with an effective amount of a composition comprising a biological active agent prior to implantation into the subject.

11. The method of claim 10, wherein the composition is an immediate release composition capable of facilitating vascularization and integration of the three-dimensional cardiac construct into the in vivo cardiac tissue of the subject.

12. The method of claim 10, wherein the composition is a time release composition capable of facilitating vascularization and integration of the three-dimensional cardiac construct into the in vivo cardiac tissue of the subject.

13. The method of claim 9, wherein the support is a suture.

14. The method of claim 13, wherein the suture is fabricated from one or more materials selected from the group consisting of silk, polypropylene, polyamide, polyvinylidene, polyester, polyether, polydioxanone, nylon, linen, cotton, plain gut, chromic gut, poliglecaprone, polyglactin, polylactide, collagen, or naturally occurring protein, and any combination thereof.

15. The method of claim 13, wherein the cardiac cells coating the suture are at a density of about 1×10⁶ cells/ml.

16. A three-dimensional vascularized cardiac construct, said construct comprising:
cardiac cells; and
a support;
wherein the cardiac cells are arranged on the support at a density of about 1×10⁶ thereby forming a three-dimensional vascularized cardiac construct having physiological characteristics similar to intact in vivo cardiac tissue.

17. The construct of claim 16, wherein the support is one or more materials selected from the group consisting of sutures, meshes, foams, gels, ceramics, acellularized extra-cellular matrix material.

18. The construct of claim 17, wherein the suture fabricated from one or more components selected from the group consisting of silk, polypropylene, polyamide, polyvinylidene, polyester, polyether, polydioxanone, nylon, linen, cotton, plain gut, chromic gut, poliglecaprone, polyglactin, polylactide, collagen, or naturally occurring protein, and any combination thereof.

19. The construct of claim 17, wherein the cardiac cells have been genetically modified to produce one or more gene products having at least one ability selected from the group consisting of to enhance the growth of seeded cells, to enhance migration, to enhance cardiac function, to facilitate angiogenesis, and to reduce the likelihood of thrombus formation.

20. The construct of claim 17, wherein the three-dimensional cardiac construct is further coated with an effective amount of a biological active agent.

21. The construct of claim 20, wherein the biological agent is capable of promoting angiogenesis.

22. The construct of claim 21, wherein the biological agent is one or more compounds selected from the group consisting of FGF, bFGF, acid FGF (aFGF), FGF-2, FGF-4, EGF, PDGF, TGF-betal, angiopoietin-1, angiopoietin-2, PlGF, VEGF, and any combination thereof.

23. The construct of claim 20, wherein the biological agent is an antibiotic.
